(19) United States
(12) Patent Application Publication
(10) Pub. No.: US 2005/0019925 A1
(43) Pub. Date: Jan. 27, 2005

(54) Intron fusion construct and method of using for selecting high-expressing production cell lines

(75) Inventors: Lynne Ann Krummen, San Francisco, CA (US); Amy Yijuan Shen, San Mateo, CA (US); Vanessa Chisholm, San Mateo, CA (US)

Correspondence Address: Genentech, Inc., 1DNA Way, South San Francisco, CA, 94080-4990, US

(73) Assignee: Genentech, Inc., South San Francisco, CA (US)

(21) Appl. No.: 10/715,270

(22) Filed: Nov. 14, 2003

Related U.S. Application Data
(60) Provisional application No. 60/426,095, filed on Nov. 14, 2002.

(51) Int. Cl. C12N 15/63; C12N 15/87

(52) U.S. Cl. 435/455; 435/468

(57) ABSTRACT

This invention relates to a DNA construct, methods of selecting for high-expressing host cells, a method of producing a protein of interest in high yields and a method of producing eukaryotic cells having multiple copies of a sequence encoding a protein of interest. In one method, stable clones capable of producing a high level of a product of interest are generated from one step of a direct selection immediately after transfection.

This application claims priority under 35 U.S.C. § 119(e) from U.S. provisional application Ser. No. 60/426,095, filed Nov. 14, 2002, which is herein incorporated by reference.

### BACKGROUND OF THE INVENTION

1. Field of the Invention

This invention relates to a DNA construct, a method of selecting for high-expressing host cells, a method of producing a protein of interest in high yields and a method of producing eukaryotic cells having multiple copies of a sequence encoding a protein of interest.

2. Description of Background and Related Art

The discovery of methods for introducing DNA into living host cells in a functional form has provided the key to understanding many fundamental biological processes, and has made possible the production of important proteins and other molecules in commercially useful quantities.

Despite the general success of such gene transfer methods, several common problems exist that may limit the efficiency with which a gene encoding a desired protein can be introduced into and expressed in a host cell. One problem is knowing when the gene has been successfully transferred into recipient cells. A second problem is distinguishing between those cells that contain the gene and those that have survived the transfer procedures but do not contain the gene. A third problem is identifying and isolating those cells that contain the gene and that are expressing high levels of the protein encoded by the gene.

In general, the known methods for introducing genes into eukaryotic cells tend to be highly inefficient. Of the cells in a given culture, only a small proportion take up and express exogenously added DNA, and an even smaller proportion stably maintain that DNA.

Identification of those cells that have incorporated a product gene encoding a desired protein typically is achieved by introducing into the same cells another gene, commonly referred to as a selectable gene, that encodes a selectable marker. A selectable marker is a protein that is necessary for the growth or survival of a host cell under the particular culture conditions chosen, such as an enzyme that confers resistance to an antibiotic or other drug, or an enzyme that compensates for a metabolic or catabolic defect in the host cell. For example, selectable genes commonly used with eukaryotic cells include the genes for aminoglycoside phosphotransferase (APH), hygromycin phosphotransferase (hyg), dihydrofolate reductase (DHFR), thymidine kinase (tk), neomycin resistance, puromycin resistance, glutamine synthetase, and asparagine synthetase.

The method of identifying a host cell that has incorporated one gene on the basis of expression by the host cell of a second incorporated gene encoding a selectable marker is referred to as cotransfectation (or cotransfection). In that method, a gene encoding a desired polypeptide and a selection gene typically are introduced into the host cell simultaneously. In this case of simultaneous cotransfectation, the gene encoding the desired polypeptide and the selectable gene may be present on a single DNA molecule or on separate DNA molecules prior to being introduced into the host cells. Wigler et al., Cell, 16:777 (1979). Cells that have incorporated the gene encoding the desired polypeptide then are identified or isolated by culturing the cells under conditions that preferentially allow for the growth or survival of those cells that synthesize the selectable marker encoded by the selectable gene.

The level of expression of a gene introduced into a eukaryotic host cell depends on multiple factors, including gene copy number, efficiency of transcription, messenger RNA (mRNA) processing, stability, and translation efficiency. Accordingly, high level expression of a desired polypeptide typically will involve optimizing one or more of those factors.

For example, the level of protein production may be increased by covalently joining the coding sequence of the gene to a “strong” promoter or enhancer that will give high levels of transcription. Promoters and enhancers are nucleotide sequences that interact specifically with proteins in a host cell that are involved in transcription. Kriegler, Meth. Enzymol., 185:512 (1990); Maniatis et al., Science, 236:1237 (1987). Promoters are located upstream of the coding sequence of a gene and facilitate transcription of the gene by RNA polymerase. Among the eukaryotic promoters that have been identified as strong promoters for high-level expression are the SV40 early promoter, adenovirus major late promoter, mouse metallothionein-I promoter, Rous sarcoma virus long terminal repeat, and human cytomegalovirus immediate early promoter (CMV).

Enhancers stimulate transcription from a linked promoter. Unlike promoters, enhancers are active when placed downstream from the transcription initiation site or at considerable distances from the promoter, although in practice enhancers may overlap physically and functionally with promoters. For example, all of the strong promoters listed above also contain strong enhancers. Bendig, Genetic Engineering, 7:91 (Academic Press, 1988).

The level of protein production also may be increased by increasing the gene copy number in the host cell. One method for obtaining high gene copy number is to directly introduce into the host cell multiple copies of the gene, for example, by using a large molar excess of the product gene relative to the selectable gene during cotransfectation. Kaufman, Meth. Enzymol., 185:537 (1990). With this method, however, only a small proportion of the cotransfected cells will contain the product gene at high copy number. Furthermore, because no generally applicable, convenient method exists for distinguishing such cells from the majority of cells that contain fewer copies of the product gene, laborious and time-consuming screening methods typically are required to identify the desired high-copy number transfectants.

Another method for obtaining high gene copy number involves cloning the gene in a vector that is capable of replicating autonomously in the host cell. Examples of such vectors include mammalian expression vectors derived from Epstein-Barr virus or bovine papilloma virus, and yeast 2-micron plasmid vectors. Stephens & Hentschel, Biochem. J., 248:1 (1987); Yates et al., Nature, 313:812 (1985); Beggs, Genetic Engineering, 2:175 (Academic Press, 1981).

Yet another method for obtaining high gene copy number involves gene amplification in the host cell. Gene amplification occurs naturally in eukaryotic cells at a relatively low frequency. Schimke, J. Biol. Chem., 263:5989 (1988). However, gene amplification also may be induced, or at least selected for, by exposing host cells to appropriate selective pressure. For example, in many cases it is possible to introduce a product gene together with an amplifiable gene into a host cell and subsequently select for amplification of the marker gene by exposing the cotransfected cells to sequentially increasing concentrations of a selective agent. Typically the product gene will be coamplified with the marker gene under such conditions.

The most widely used amplifiable gene for that purpose is a DHFR gene, which encodes a dihydrofolate reductase enzyme. The selection conditions used in conjunction with a DHFR gene are the absence of glycine, hypoxanthine and thymidine (GHT) with or without the presence of methotrexate (Mtx). A host cell is cotransfected with a product gene encoding a desired protein and a DHFR gene, and transfectants are identified by first culturing the cells in GHT-free culture medium that may contains Mtx. A suitable host cell when a wild-type DHFR gene is used is the Chinese Hamster Ovary (CHO) cell line deficient in DHFR activity, prepared and propagated as described by Urlaub & Chasin, Proc. Nat. Acad. Sci. USA, 77:4216 (1980). The transfected cells then are exposed to successively higher amounts of Mtx. This leads to the synthesis of multiple copies of the DHFR gene, and concomitantly, multiple copies of the product gene. Schimke, J. Biol. Chem., 263:5989 (1988); Axel et al., U.S. Pat. No. 4,399,216; Axel et al., U.S. Pat. No. 4,634,665. Other references directed to co-transfection of a gene together with a genetic marker that allows for selection and subsequent amplification include Kaufman in Genetic Engineering, ed. J. Setlow (Plenum Press, New York), Vol. 9 (1987); Kaufman and Sharp, J. Mol. Biol., 159:601 (1982); Ringold et al., J. Mol. Appl. Genet., 1:165-175 (1981); Kaufman et al., Mol. Cell Biol., 5:1750-1759 (1985); Kaetzel and Nilson, J. Biol. Chem., 263:6244-6251 (1988); Hung et al., Proc. Natl. Acad. Sci. USA, 83:261-264 (1986); Kaufman et al., EMBO J., 6:87-93 (1987); Johnston and Kucey, Science, 242:1551-1554 (1988); Urlaub et al., Cell, 33:405-412 (1983).

To extend the DHFR amplification method to other cell types, a mutant DHFR gene that encodes a protein with reduced sensitivity to methotrexate may be used in conjunction with host cells that contain normal numbers of an endogenous wild-type DHFR gene. Simonsen and Levinson, Proc. Natl. Acad. Sci. USA, 80:2495 (1983); Wigler et al., Proc. Natl. Acad. Sci. USA, 77:3567-3570 (1980); Haber and Schimke, Somatic Cell Genetics, 8:499-508 (1982).

Alternatively, host cells may be co-transfected with the product gene, a DHFR gene, and a dominant selectable gene, such as a neo gene. Kim and Wold, Cell, 42:129 (1985); Capon et al., U.S. Pat. No. 4,965,199. Transfectants are identified by first culturing the cells in culture medium containing neomycin (or the related drug G418), and the transfectants so identified then are selected for amplification of the DHFR gene and the product gene by exposure to successively increasing amounts of Mtx.

As will be appreciated from this discussion, the selection of recombinant host cells that express high levels of a desired protein generally is a multi-step process. In the first step, initial transfectants are selected that have incorporated the product gene and the selectable gene. In subsequent steps, the initial transfectants are subject to further selection for high-level expression of the selectable gene and then random screening for high-level expression of the product gene. To identify cells expressing high levels of the desired protein, typically one must screen large numbers of transfectants. The majority of transfectants produce less than maximal levels of the desired protein. Further, Mtx resistance in DHFR transformants is at least partially conferred by varying degrees of gene amplification. Schimke, Cell, 37:705-713 (1984). The inadequacies of co-expression of the non-selected gene have been reported by Wold et al., Proc. Natl. Acad. Sci. USA, 76:5684-5688 (1979). Instability of the amplified DNA is reported by Kaufman and Schimke, Mol. Cell Biol., 1:1069-1076 (1981); Haber and Schimke, Cell, 26:355-362 (1981); and Fedespiel et al., J. Biol. Chem., 259:9127-9140 (1984).

Several methods have been described for directly selecting such recombinant host cells in a single step. One strategy involves co-transfecting host cells with a product gene and a DHFR gene, and selecting those cells that express high levels of DHFR by directly culturing in medium containing a high concentration of Mtx. Many of the cells selected in that manner also express the co-transfected product gene at high levels Page and Sydenham, Bio/Technology, 9:64 (1991). This method for single-step selection suffers from certain drawbacks that limit its usefulness. High-expressing cells obtained by direct culturing in medium containing a high level of a selection agent may have poor growth and stability characteristics, thus limiting their usefulness for long-term production processes Page and Snyderman, Bio/Technology, 9:64 (1991). Single-step selection for high-level resistance to Mtx may produce cells with an altered, Mtx-resistant DHFR enzyme, or cells that have altered Mtx transport properties, rather than cells containing amplified genes. Haber et al., J. Biol. Chem., 256:9501 (1981); Assaraf and Schimke, Proc. Natl. Acad. Sci. USA, 84:7154 (1987).

Another method involves the use of polycistronic mRNA expression vectors containing a product gene at the 5′ end of the transcribed region and a selectable gene at the 3′ end. Because translation of the selectable gene at the 3′ end of the polycistronic mRNA is inefficient, such vectors exhibit preferential translation of the product gene and require high levels of polycistronic mRNA to survive selection. Kaufman, Meth. Enzymol., 185:487 (1990); Kaufman, Meth. Enzymol., 185:537 (1990); Kaufman et al., EMBO J., 6:187 (1987). Accordingly, cells expressing high levels of the desired protein product may be obtained in a single step by culturing the initial transfectants in medium containing a selection agent appropriate for use with the particular selectable gene. However, the utility of these vectors is variable because of the unpredictable influence of the upstream product reading frame on selectable marker translation and because the upstream reading frame sometimes becomes deleted during methotrexate amplification (Kaufman et al., J. Mol. Biol., 159:601-621 (1982); Levinson, Methods in Enzymology, San Diego: Academic Press, Inc. (1990)). Later vectors incorporated an internal translation initiation site derived from members of the picornavirus family which is positioned between the product gene and the selectable gene (Pelletier et al., Nature, 334:320 (1988); Jang et al., J. Virol., 63:1651 (1989)).

A third method for single-step selection involves use of a DNA construct with a selectable gene containing an intron within which is located a gene encoding the protein of interest. See U.S. Pat. No. 5,043,270 and Abrams et al., J. Biol. Chem., 264(24): 14016-14021 (1989). In yet another single-step selection method, host cells are co-transfected with an intron-modified selectable gene and a gene encoding the protein of interest. See WO 92/17566, published Oct. 15, 1992. The intron-modified gene is prepared by inserting into the transcribed region of a selectable gene an intron of such length that the intron is correctly spliced from the corresponding mRNA precursor at low efficiency, so that the amount of selectable marker produced from the intron-modified selectable gene is substantially less than that produced from the starting selectable gene. These vectors help to insure the integrity of the integrated DNA construct, but transcriptional linkage is not achieved as selectable gene and the protein gene are driven by separate promoters.

Other mammalian expression vectors that have single transcription units have been described. Retroviral vectors have been constructed (Cepko et al., Cell, 37:1053-1062 (1984)) in which a cDNA is inserted between the endogenous Moloney murine leukemia virus (M-MuLV) splice donor and splice acceptor sites which are followed by a neomycin resistance gene. This vector has been used to express a variety of gene products following retroviral infection of several cell types.

A method for selecting recombinant host cells expressing high levels of a desired protein was previously described by the applicants in Lucas et al., Nucleic Acid Research, 24, No. 9: 1774-1779 and U.S. Pat. No. 5,561,053. That method utilizes eukaryotic host cells harboring a DNA construct comprising a selectable gene (preferably an amplifiable gene) and a product gene provided 3′ to the selectable gene. The selectable gene is positioned within an intron defined by a splice donor site and a splice acceptor site and the selectable gene and product gene are under the transcriptional control of a single transcriptional regulatory region. The splice donor site is generally an efficient splice donor site and thereby regulates expression of the product gene using the transcriptional regulatory region. The transfected cells are cultured so as to express the gene encoding the product in a selective medium which may contain an amplifying agent for sufficient time to allow cells having multiple copies of the product gene, or cells with a single (or multiple) copy of the gene in a chromosomal loci with high transcriptional activity to be identified.

Other fusion expression constructs have been developed. For example, a fusion of green flourescent protein with the Zeocin-resistance marker construct has been created. Bennet, R. P. et al., Biotechniques. 24(3):478-82, 1998 March. Such constructs were used to allow visual screening and drug selection of transfected eukaryotic cells.

In another example, human prothrombin was overexpressed in transformed eukaryotic cells using a dominant bifunctional selection and amplification marker. Herlitschka, Sabine E. et al., Protein Expression and Purification. 8, 358-364, 1996 July. In this reference the marker consisted of the murine wild-type dihydrofolate reductase cDNA and the E. coli hygromycin phosphotransferase gene fused in frame. The gene of interest is connected, upstream, by the EMCV untranslated region to the fusion marker gene, forming a dicistronic transcription unit.

With the state of the art in mind, it is one object of the present invention to increase the level of homogeneity with regard to expression levels of stable clones transfected with a product gene of interest, by expressing fused selectable markers (i.e. DHFR and puromycin) and a protein of interest from a single promoter.

It is another object to provide a method for selecting stable, recombinant host cells that express high levels of a desired protein product, which method is rapid and convenient to perform, and reduces the numbers of transfected cells which need to be screened. Furthermore, it is an object to allow high levels of single and multiple unit polypeptides to be rapidly generated from clones or pools of stable host cell transfectants.

It is an additional object to provide expression vectors which bias for active integration events (i.e. have an increased tendency to generate transformants wherein the DNA construct is inserted into a region of the genome of the host cell which results in high level expression of the product gene) and can accommodate a variety of product genes without the need for modification.

### SUMMARY OF THE INVENTION

Accordingly, the present invention is directed to a DNA construct (DNA molecule) comprising a 5′ transcriptional initiation site and a 3′ transcriptional termination site, two selectable genes that have been fused into one open reading frame (preferably amplifiable genes) and a product gene provided 3′ to the fused selectable genes, a transcriptional regulatory region regulating transcription of both the fused selectable genes and the product gene, the fused selectable genes positioned within an intron defined by a splice donor site and a splice acceptor site. The splice donor site preferably comprises an effective splice donor sequence as herein defined and thereby regulates expression of the product gene using the transcriptional regulatory region.

In another embodiment, the invention provides a method for producing a product of interest comprising culturing a eukaryotic cell which has been transfected with the DNA construct described above, so as to express the product gene and recovering the product.

In a further embodiment, the invention provides a method for producing eukaryotic cells having multiple copies of the product gene comprising transfecting eukaryotic cells with the DNA construct described above (where the selectable fused genes are amplifiable genes), growing the cells in a selective medium comprising an amplifying agent(s) for a sufficient time for amplification to occur, and selecting cells having multiple copies of the product gene. After transfection of the host cells, most of the transfectants fail to exhibit the selectable phenotype characteristic of the protein encoded by either of the selectable genes, but surprisingly a small proportion of the transfectants do exhibit one or both of the selectable phenotype, and among those transfectants, the majority are found to express high levels of the desired product encoded by the product gene. Thus, the invention provides an improved method for the selection of recombinant host cells expressing high levels of a desired product, which method is useful with a wide variety of eukaryotic host cells and avoids the problems inherent in, and improves upon, existing cell selection technology.

### BRIEF DESCRIPTION OF THE DRAWINGS

FIG. 1 illustrates schematically the construction of the pSV.IPD. The gene for the protein of interest would be inserted at the polylinker site.

FIGS. 2-1 to 2-4 depict the nucleotide sequence of the pSV.IPUR plasmid used in constructing pSV.IPD (SEQ ID NO 1).

FIGS. 3-1 to 3-4 depict the nucleotide sequence of the pSV.ID plasmid used in constructing pSV.IPD (SEQ ID NO 2).

FIGS. 4-1 to 4-4 depict the nucleotide sequence of the pSV.IPD (SEQ ID NO 3).

FIG. 5 illustrates schematically the plasmid, pSV.ID.VEGF, used as a control in Example 1.

FIG. 6 illustrates schematically the plasmid, pSV.IPD.2C4, used in Example 1 (SEQ ID NO 4).

FIGS. 7-1 to 7-8 depict the nucleotide sequence of the pSV.IPD.2C4 plasmid used in Example 1.

FIG. 8 depicts a FACS analysis of transiently transfected CHO cells with a GTP plasmid in 250 ml spinner transfection. FACS analysis was performed 24 hours after transfection.

FIG. 9 depicts the expression level of clones from traditional 10 nM MTX selection. Cells were transfected with commercial transfection reagent and directly selected in 10 nM MTX. Individual clones were grown in a 96-well plate. Product accumulated for 6 days prior to ELISA.

FIGS. 10-1 and 10-2 depict the expression level of clones from 25 and 50 nM MTX direct selections, respectively, of SV40-based constructs derived from spinner transfection. The assay was performed the same as in FIG. 9.

FIG. 11 depicts the expression level of clones from 25 nM MTX direct selection of CMV-based construct derived from spinner transfection. The assay was performed the same as in FIG. 9.

FIG. 12 depicts the titer evaluation in Miniferm. Samples were collected every day and submitted to an HPLC protein A assay for titer.

FIG. 13-1 to 13-7 depict the nucleotide sequence of the pCMV.IPD.Heterologous polypeptide (HP) plasmid used in Example 3.

FIG. 14-1 to 14-8 depicts the nucleotide sequence of the pSV40.IPD.HP plasmid used in Example 3.

FIG. 15 illustrates schematically the plasmid, pCMV.IPD.HP, used in Example 3.

FIG. 16 illustrates a time line and titer comparison between a traditional selection and direct selection method described in Example 3. Equivalent titers are indicated horizontally across the illustration. For example, the titers for a 200/300 nM SV40-plasmid traditional selection, 100 nM SV40-plasmid direct selection and 25 nm CMV-plasmid direct selection are roughly equivalent.

### DESCRIPTION OF THE PREFERRED EMBODIMENT

Definitions:

The “DNA construct” disclosed herein comprises a non-naturally occurring DNA molecule or chemical analog which can either be provided as an isolate or integrated in another DNA molecule e.g in an expression vector or the chromosome of an eukaryotic host cell.

The term “selectable gene” as used herein refers to a DNA that encodes a selectable marker necessary for the growth or survival of a host cell under the particular cell culture conditions chosen. Accordingly, a host cell that is transformed with a selectable gene will be capable of growth or survival under certain cell culture conditions wherein a non-transfected host cell is not capable of growth or survival. Typically, a selectable gene will confer resistance to a drug or compensate for a metabolic or catabolic defect in the host cell. Examples of selectable genes are provided in the following table. See also Kaufman, Methods in Enzymology, 185: 537-566 (1990), for a review of these.

“Fused selectable genes” as used herein refers to a DNA that encodes at least two selectable markers in the same open reading frame and inserted into an intron sequence.

TABLE 1
Examples of Selectable Genes and their Selection Agents

| Selection Agent | Selectable Gene |
|---|---|
| Puromycin | Puromycin-N-acetyltransferase |
| Methotrexate | Dihydrofolate reductase |
| Cadmium | Metallothionein |
| PALA | CAD |
| Xyl-A-or adenosine and 2′- | Adenosine deaminase |
| deoxycoformycin | |
| Adenine, azaserine, and coformycin | Adenylate deaminase |
| 6-Azauridine, pyrazofuran | UMP Synthetase |
| Mycophenolic acid | IMP 5′-dehydrogenase |
| Mycophenolic acid with limiting | Xanthine-guanine |
| xanthine | phosphoribosyltransferase |
| Hypoxanthine, aminopterin, and | Mutant HGPRTase or mutant |
| thymidine (HAT) | thymidine kinase |
| 5-Fluorodeoxyuridine | Thymidylate synthetase |
| Multiple drugs e.g. adriamycin, | P-glycoprotein 170 |
| vincristine or colchicine | |
| Aphidicolin | Ribonucleotide reductase |
| Methionine sulfoximine | Glutamine synthetase |
| β-Aspartyl hydroxamate or Albizziin | Asparagine synthetase |
| Canavanine | Arginosuccinate synthetase |
| α-Difluoromethylornithine | Ornithine decarboxylase |
| Compactin | HMG-CoA reductase |
| Tunicamycin | N-Acetylglucosaminyl transferase |
| Borrelidin | Threonyl-tRNA synthetase |
| Ouabain | Na⁺K⁺-ATPase |

The preferred selectable genes are amplifiable genes. As used herein, the term “amplifiable gene” refers to a gene which is amplified (i.e. additional copies of the gene are generated which survive in intrachromosomal or extrachromosomal form) under certain conditions. The amplifiable gene(s) usually encodes an enzyme (i.e. an amplifiable marker) which is required for growth of eukaryotic cells under those conditions. For example, the gene may encode DHFR which is amplified when a host cell transformed therewith is grown in Mtx. According to Kaufman, the selectable genes in Table 1 above can also be considered amplifiable genes. An example of a selectable gene which is generally not considered to be an amplifiable gene is the neomycin resistance gene (Cepko et al., supra).

As used herein, “selective medium” refers to nutrient solution used for growing eukaryotic cells which have the selectable gene(s) and therefore is deficient in components supplied by the selectable gene or includes a “selection agent”. Commercially available media based on formulations such as Ham's F10 (Sigma), Minimal Essential Medium ((MEM), Sigma), RPMI-1640 (Sigma), and Dulbecco's Modified Eagle's Medium ((DMEM), Sigma) are exemplary nutrient solutions. In addition, any of the media described in Ham and Wallace, Meth. Enz., 58:44 (1979), Barnes and Sato, Anal. Biochem., 102:255 (1980), U.S. Pat. Nos. 4,767,704; 4,657,866; 4,927,762; or U.S. Pat. No. 4,560,655; WO 90/03430; WO 87/00195; U.S. Pat. No. Re. 30,985; or U.S. Pat. No. 5,122,469, the disclosures of all of which are incorporated herein by reference, may be used as culture media. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The preferred nutrient solution comprises fetal bovine serum.

The term “selection agent” refers to a substance that interferes with the growth or survival of a host cell possibly because the cell is deficient in a particular selectable gene. Examples of selection agents are presented in Table 1 above. The selection agent preferably comprises an “amplifying agent” which is defined for purposes herein as an agent for amplifying copies of the amplifiable gene or causing integration of multiple copies of the amplifiable gene into the genome, such as Mtx if the amplifiable gene is DHFR. See Table 1 for examples of amplifying agents.

As used herein, the terms “direct selection” or “direct culturing” means the first exposure to selective conditions either without MTX or GHT or with MTX, and production of a heterologous polypeptide in an amount of about 250 mg/l, 400 mg/l, 600 mg/l or 800 mg/l up to about 1 g/l or more.

As used herein, the term “transcriptional initiation site” refers to the nucleic acid in the DNA construct corresponding to the first nucleic acid incorporated into the primary transcript, i.e., the mRNA precursor, which site is generally provided at, or adjacent to, the 5′ end of the DNA construct.

The term “transcriptional termination site” refers to a sequence of DNA, normally represented at the 3′ end of the DNA construct, that causes RNA polymerase to terminate transcription.

As used herein, “transcriptional regulatory region” refers to a region of the DNA construct that regulates transcription of the selectable gene and the product gene. The transcriptional regulatory region normally refers to a promoter sequence (i.e. a region of DNA involved in binding of RNA polymerase to initiate transcription) which can be constitutive or inducible and, optionally, an enhancer (i.e. a cis-acting DNA element, usually from about 10-300 bp, that acts on a promoter to increase its transcription).

As used herein, “product gene” refers to DNA that encodes a desired protein or polypeptide product. Any product gene that is capable of expression in a host cell may be used, although the methods of the invention are particularly suited for obtaining high-level expression of a product gene that is not also a selectable or amplifiable gene. Accordingly, the protein or polypeptide encoded by a product gene typically will be one that is not necessary for the growth or survival of a host cell under the particular cell culture conditions chosen. For example, product genes suitably encode a peptide, or may encode a polypeptide sequence of amino acids for which the chain length is sufficient to produce higher levels of tertiary and/or quaternary structure.

Examples of bacterial polypeptides or proteins include, e.g., alkaline phosphatase and β-lactamase. Examples of mammalian polypeptides or proteins include molecules such as renin; a growth hormone, including human growth hormone, and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; antibodies; chimeric proteins such as immunoadhesins and fragments of any of the above-listed polypeptides.

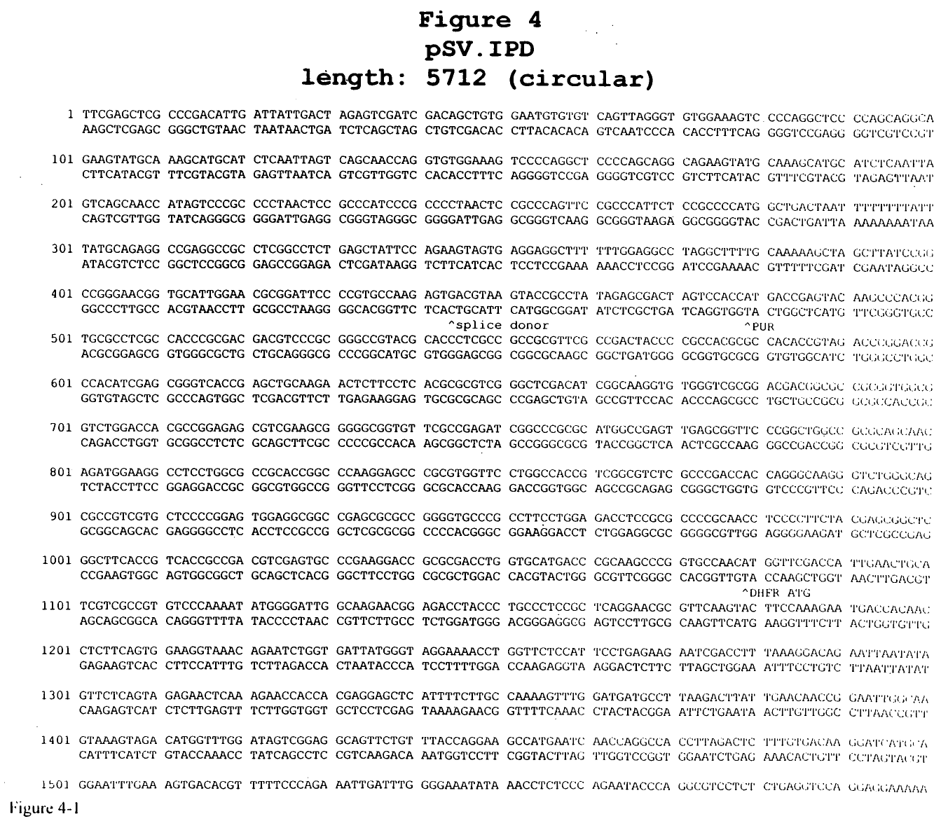

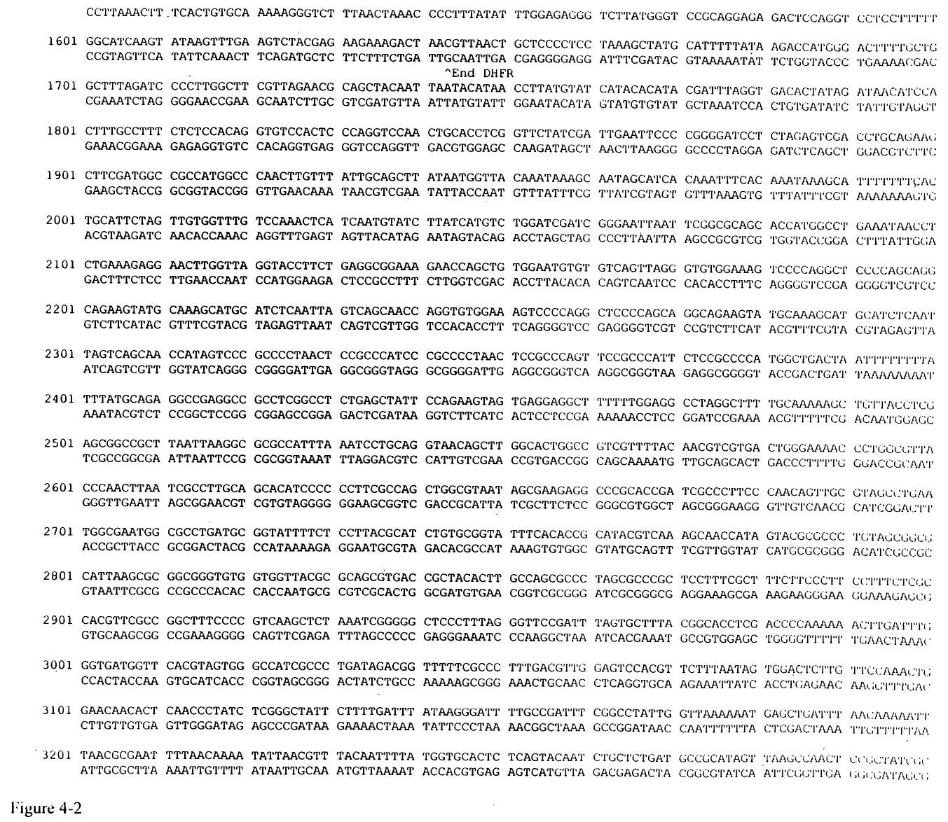

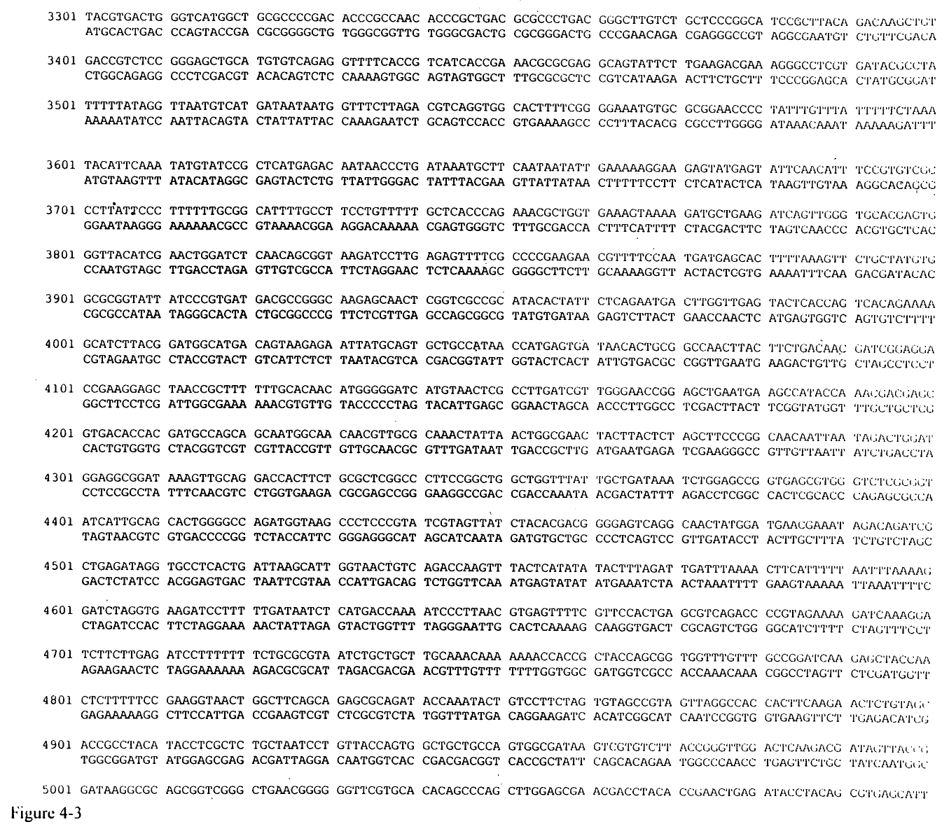

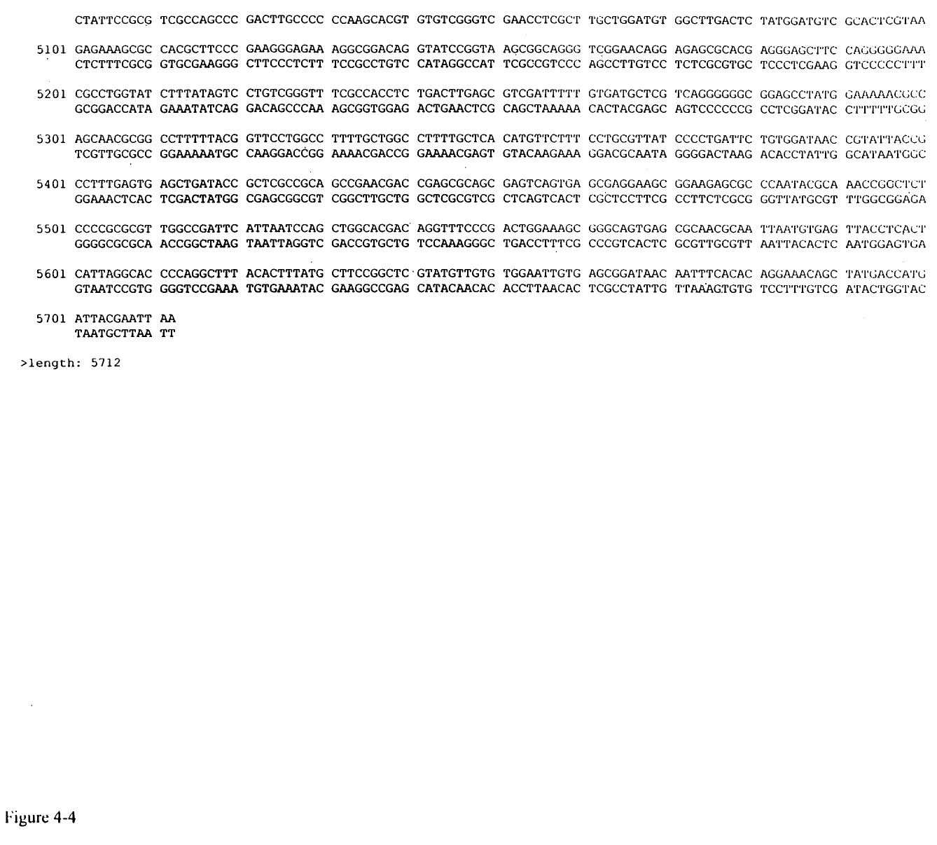

The product gene preferably does not consist of an anti-sense sequence for inhibiting the expression of a gene present in the host. Preferred proteins herein are therapeutic proteins such as TGF-β, TGF-α, PDGF, EGF, FGF, IGF-I, DNase, plasminogen activators such as t-PA, clotting factors such as tissue factor and factor VIII, hormones such as relaxin and insulin, cytokines such as IFN-γ, chimeric proteins such as TNF receptor IgG immunoadhesin (TNFr-IgG) or antibodies such as anti-IgE. An example of an antibody that can be produced with the pSV.IDP plasmid (FIG. 4) is anti-HER2 Neu antibody, 2C4, as provided in Example 1, supra.

The term “intron” as used herein refers to a nucleotide sequence present within the transcribed region of a gene or within a messenger RNA precursor, which nucleotide sequence is capable of being excised, or spliced, from the messenger RNA precursor by a host cell prior to translation. Introns suitable for use in the present invention are suitably prepared by any of several methods that are well known in the art, such as purification from a naturally occurring nucleic acid or de novo synthesis. The introns present in many naturally occurring eukaryotic genes have been identified and characterized. Mount, Nuc. Acids Res., 10:459 (1982). Artificial introns comprising functional splice sites also have been described. Winey et al., Mol. Cell Biol., 9:329 (1989); Gatermann et al., Mol. Cell Biol., 9:1526 (1989). Introns may be obtained from naturally occurring nucleic acids, for example, by digestion of a naturally occurring nucleic acid with a suitable restriction endonuclease, or by PCR cloning using primers complementary to sequences at the 5′ and 3′ ends of the intron. Alternatively, introns of defined sequence and length may be prepared synthetically using various methods in organic chemistry. Narang et al., Meth. Enzymol., 68:90 (1979); Caruthers et al., Meth. Enzymol., 154:287 (1985); Froehler et al., Nuc. Acids Res., 14:5399 (1986).

As used herein “splice donor site” or “SD” refers to the DNA sequence immediately surrounding the exon-intron boundary at the 5′ end of the intron, where the “exon” comprises the nucleic acid 5′ to the intron. Many splice donor sites have been characterized and Ohshima et al., J. Mol. Biol., 195:247-259 (1987) provides a review of these. An “efficient splice donor sequence” refers to a nucleic acid sequence encoding a splice donor site wherein the efficiency of splicing of messenger RNA precursors having the splice donor sequence is between about 80 to 99% and preferably 90 to 95% as determined by quantitative PCR. Examples of efficient splice donor sequences include the wild type (WT) ras splice donor sequence and the GAC:GTAAGT sequence of Example 3. Other efficient splice donor sequences can be readily selected using the techniques for measuring the efficiency of splicing disclosed herein.

The terms “PCR” and “polymerase chain reaction” as used herein refer to the in vitro amplification method described in U.S. Pat. No. 4,683,195 (issued Jul. 28, 1987). In general, the PCR method involves repeated cycles of primer extension synthesis, using two DNA primers capable of hybridizing preferentially to a template nucleic acid comprising the nucleotide sequence to be amplified. The PCR method can be used to clone specific DNA sequences from total genomic DNA, cDNA transcribed from cellular RNA, viral or plasmid DNAs. Wang & Mark, in PCR Protocols, pp. 70-75 (Academic Press, 1990); Scharf, in PCR Protocols, pp. 84-98; Kawasaki & Wang, in PCR Technology, pp. 89-97 (Stockton Press, 1989). Reverse transcription-polymerase chain reaction (RT-PCR) can be used to analyze RNA samples containing mixtures of spliced and unspliced mRNA transcripts. Fluorescently tagged primers designed to span the intron are used to amplify both spliced and unspliced targets. The resultant amplification products are then separated by gel electrophoresis and quantitated by measuring the fluorescent emission of the appropriate band(s). A comparison is made to determine the amount of spliced and unspliced transcripts present in the RNA sample.

One preferred splice donor sequence is a “consensus splice donor sequence”. The nucleotide sequences surrounding intron splice sites, which sequences are evolutionarily highly conserved, are referred to as “consensus splice donor sequences”. In the mRNAs of higher eukaryotes, the 5′ splice site occurs within the consensus sequence AG:GUAAGU (wherein the colon denotes the site of cleavage and ligation). In the mRNAs of yeast, the 5′ splice site is bounded by the consensus sequence :GUAUGU. Padgett, et al., Ann. Rev. Biochem., 55:1119 (1986).

The expression “splice acceptor site” or “SA” refers to the sequence immediately surrounding the intron-exon boundary at the 3′ end of the intron, where the “exon” comprises the nucleic acid 3′ to the intron. Many splice acceptor sites have been characterized and Ohshima et al., J. Mol. Biol., 195:247-259 (1987) provides a review of these. The preferred splice acceptor site is an efficient splice acceptor site which refers to a nucleic acid sequence encoding a splice acceptor site wherein the efficiency of splicing of messenger RNA precursors having the splice acceptor site is between about 80 to 99% and preferably 90 to 95% as determined by quantitative PCR. The splice acceptor site may comprise a consensus sequence. In the mRNAs of higher eukaryotes, the 3′ splice acceptor site occurs within the consensus sequence (U/C)₁₁NCAG:G. In the mRNAs of yeast, the 3′ acceptor splice site is bounded by the consensus sequence (C/U)AG:. Padgett, et al., supra.

As used herein “culturing for sufficient time to allow amplification to occur” refers to the act of physically culturing the eukaryotic host cells which have been transformed with the DNA construct in cell culture media containing the amplifying agent, until the copy number of the amplifiable gene (and preferably also the copy number of the product gene) in the host cells has increased relative to the transformed cells prior to this culturing.

The term “expression” as used herein refers to transcription or translation occurring within a host cell. The level of expression of a product gene in a host cell may be determined on the basis of either the amount of corresponding mRNA that is present in the cell or the amount of the protein encoded by the product gene that is produced by the cell. For example, mRNA transcribed from a product gene is desirably quantitated by northern hybridization or quantitative real-time PCR. Sambrook, et al., Molecular Cloning: A Laboratory Manual, pp. 7.3-7.57 (Cold Spring Harbor Laboratory Press, 1989). Protein encoded by a product gene can be quantitated either by assaying for the biological activity of the protein or by employing assays that are independent of such activity, such as western blotting or radioimmunoassay using antibodies that are capable of reacting with the protein. Sambrook, et al., Molecular Cloning: A Laboratory Manual, pp. 18.1-18.88 (Cold Spring Harbor Laboratory Press, 1989).

Modes for Carrying Out the Invention

Methods and compositions are provided for enhancing the stability and/or copy number of a transcribed sequence in order to allow for elevated levels of a RNA sequence of interest. In general, the methods of the present invention involve transfecting a eukaryotic host cell with an expression vector comprising both a product gene encoding a desired polypeptide and fused selectable genes.

Selectable genes and product genes may be obtained from genomic DNA, cDNA transcribed from cellular RNA, or by in vitro synthesis. For example, libraries are screened with probes (such as antibodies or oligonucleotides of about 20-80 bases) designed to identify the selectable gene or the product gene (or the protein(s) encoded thereby). Screening the cDNA or genomic library with the selected probe may be conducted using standard procedures as described in chapters 10-12 of Sambrook et al., Molecular Cloning: A Laboratory Manual (New York: Cold Spring Harbor Laboratory Press, 1989). An alternative means to isolate the selectable gene or product gene is to use PCR methodology as described in section 14 of Sambrook et al., supra.

A preferred method of practicing this invention is to use carefully selected oligonucleotide sequences to screen cDNA libraries from various tissues known to contain the selectable gene or product gene. The oligonucleotide sequences selected as probes should be of sufficient length and sufficiently unambiguous that false positives are minimized.

The oligonucleotide generally is labeled such that it can be detected upon hybridization to DNA in the library being screened. The preferred method of labeling is to use ³²P-labeled ATP with polynucleotide kinase, as is well known in the art, to radiolabel the oligonucleotide. However, other methods may be used to label the oligonucleotide, including, but not limited to, biotinylation or enzyme labeling.

Sometimes, the DNA encoding the fused selectable genes and product gene is preceded by DNA encoding a signal sequence having a specific cleavage site at the N-terminus of the mature protein or polypeptide. In general, the signal sequence may be a component of the expression vector, or it may be a part of the selectable gene or product gene that is inserted into the expression vector. If a heterologous signal sequence is used, it preferably is one that is recognized and processed (i.e., cleaved by a signal peptidase) by the host cell. For yeast secretion the native signal sequence may be substituted by, e.g., the yeast invertase leader, alpha factor leader (including Saccharomyces and Kluyveromyces α-factor leaders, the latter described in U.S. Pat. No. 5,010,182 issued 23 Apr. 1991), or acid phosphatase leader, the C. albicans glucoamylase leader (EP 362,179 published 4 Apr. 1990), or the signal described in WO 90/13646 published 15 Nov. 1990. In mammalian cell expression the native signal sequence of the protein of interest is satisfactory, although other mammalian signal sequences may be suitable, such as signal sequences from secreted polypeptides of the same or related species, as well as viral secretory leaders, for example, the herpes simplex gD signal. The DNA for such precursor region is ligated in reading frame to the fused selectable genes or product gene.

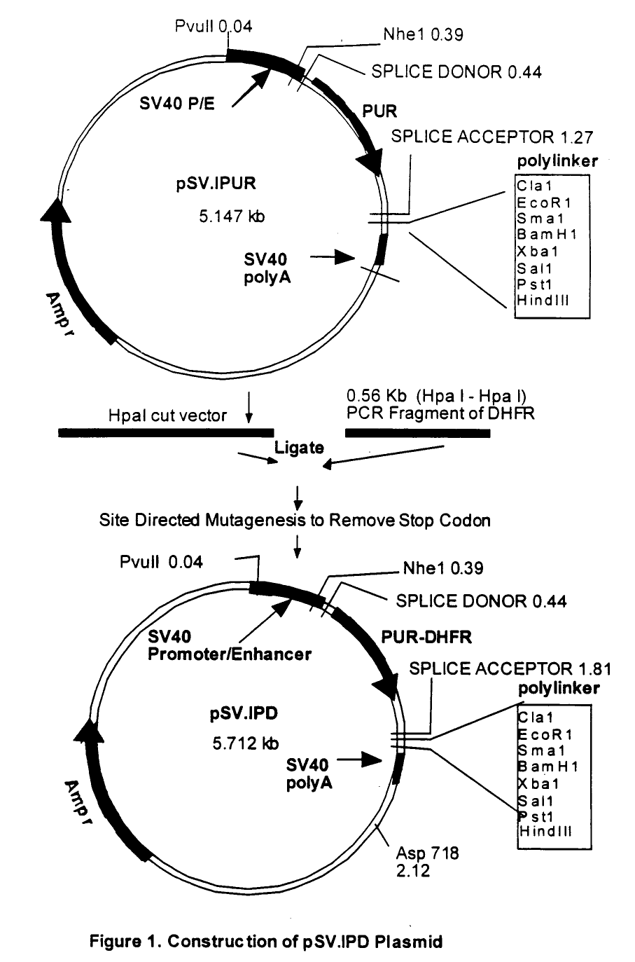

As shown in FIG. 1, the fused selectable genes are generally provided at the 5′ end of the DNA construct and are followed by the product gene (which would be inserted into the linker site). Therefore, the full-length (non-spiced) message will contain, for example, the PURO-DHFR fusion as the first open reading frame and will therefore generate PURO-DHFR protein to allow selection of stable transfectants. The full length message is not expected to generate appreciable amounts of the protein of interest as the second AUG in a dicistronic message is an inefficient initiator of translation in mammalian cells (Kozak, J. Cell Biol., 115: 887-903 (1991)).

The fused selectable genes are positioned within an intron. Introns are noncoding nucleotide sequences, normally present within many eukaryotic genes, which are removed from newly transcribed mRNA precursors in a multiple-step process collectively referred to as splicing.

A single mechanism is thought to be responsible for the splicing of mRNA precursors in mammalian, plant, and yeast cells. In general, the process of splicing requires that the 5′ and 3′ ends of the intron be correctly cleaved and the resulting ends of the mRNA be accurately joined, such that a mature mRNA having the proper reading frame for protein synthesis is produced. Analysis of a variety of naturally occurring and synthetically constructed mutant genes has shown that nucleotide changes at many of the positions within the consensus sequences at the 5′ and 3′ splice sites have the effect of reducing or abolishing the synthesis of mature mRNA. Sharp, Science, 235:766 (1987); Padgett, et al., Ann. Rev. Biochem., 55:1119 (1986); Green, Ann. Rev. Genet., 20:671 (1986). Mutational studies also have shown that RNA secondary structures involving splicing sites can affect the efficiency of splicing. Solnick, Cell, 43:667 (1985); Konarska, et al., Cell, 42:165 (1985).

The length of the intron may also affect the efficiency of splicing. By making deletion mutations of different sizes within the large intron of the rabbit beta-globin gene, Wieringa, et al. determined that the minimum intron length necessary for correct splicing is about 69 nucleotides. Cell, 37:915 (1984). Similar studies of the intron of the adenovirus E1A region have shown that an intron length of about 78 nucleotides allows correct splicing to occur, but at reduced efficiency. Increasing the length of the intron to 91 nucleotides restores normal splicing efficiency, whereas truncating the intron to 63 nucleotides abolishes correct splicing. Ulfendahl, et al., Nuc. Acids Res., 13:6299 (1985).

To be useful in the invention, the intron must have a length such that splicing of the intron from the mRNA is efficient. The preparation of introns of differing lengths is a routine matter, involving methods well known in the art, such as de novo synthesis or in vitro deletion mutagenesis of an existing intron. Typically, the intron will have a length of at least about 150 nucleotides, since introns which are shorter than this tend to be spliced less efficiently. The upper limit for the length of the intron can be up to 30 kB or more. However, as a general proposition, the intron is generally less than about 10 kB in length.

The intron is modified to contain the fused selectable genes not normally present within the intron using any of the various known methods for modifying a nucleic acid in vitro. Typically, the fused selectable genes will be introduced into an intron by first cleaving the intron with a restriction endonuclease, and then covalently joining the resulting restriction fragments to the fused selectable genes in the correct orientation for host cell expression, for example by ligation with a DNA ligase enzyme.

The DNA construct is dicistronic, i.e. the fused selectable genes and product gene are both under the transcriptional control of a single transcriptional regulatory region. As mentioned above, the transcriptional regulatory region comprises a promoter. Suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Reg., 7:149 (1968); and Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in Hitzeman et al., EP 73,657A. Yeast enhancers also are advantageously used with yeast promoters.

Expression control sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CXCAAT region where X may be any nucleotide.

Product gene transcription from vectors in mammalian host cells is controlled by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, a retrovirus, hepatitis-B virus and most preferably Simian Virus 40 (SV40) or cytomegalovirus (CMV), from heterologous mammalian promoters, e.g. the actin promoter or an immunoglobulin promoter, from heat-shock promoters, and from the promoter normally associated with the product gene, provided such promoters are compatible with the host cell systems. Promoters endogenous to the host cell system, such as the CHO Elongation Factor 1 alpha promoter may also be used.

The early and late promoters of the SV40 virus are conveniently obtained as an SV40 restriction fragment that also contains the SV40 viral origin of replication. Fiers et al., Nature, 273:113 (1978); Mulligan and Berg, Science, 209:1422-1427 (1980); Pavlakis et al., Proc. Natl. Acad. Sci. USA, 78:7398-7402 (1981). The immediate early promoter of the human cytomegalovirus (CMV) is conveniently obtained as a HindIII E restriction fragment. Greenaway et al., Gene, 18:355-360 (1982). A system for expressing DNA in mammalian hosts using the bovine papilloma virus as a vector is disclosed in U.S. Pat. No. 4,419,446. A modification of this system is described in U.S. Pat. No. 4,601,978. See also Gray et al., Nature, 295:503-508 (1982) on expressing cDNA encoding immune interferon in monkey cells; Reyes et al., Nature, 297:598-601 (1982) on expression of human β-interferon cDNA in mouse cells under the control of a thymidine kinase promoter from herpes simplex virus, Canaani and Berg, Proc. Natl. Acad. Sci. USA, 79:5166-5170 (1982) on expression of the human interferon β1 gene in cultured mouse and rabbit cells, and Gorman et al., Proc. Natl. Acad. Sci. USA, 79:6777-6781 (1982) on expression of bacterial CAT sequences in CV-1 monkey kidney cells, chicken embryo fibroblasts, Chinese hamster ovary cells, HeLa cells, and mouse NIH-3T3 cells using the Rous sarcoma virus long terminal repeat as a promoter.

Preferably the transcriptional regulatory region in higher eukaryotes comprises an enhancer sequence. Enhancers are relatively orientation and position independent having been found 5′ (Lainins et al., Proc. Natl. Acad. Sci. USA, 78:993 (1981)) and 3′ (Lusky et al., Mol. Cell Bio., 3:1108 (1983)) to the transcription unit, within an intron (Banerji et al., Cell, 33:729 (1983)) as well as within the coding sequence itself (Osborne et al., Mol. Cell Bio., 4:1293 (1984)). Many enhancer sequences are now known from mammalian genes (globin, elastase, albumin, α-fetoprotein and insulin). Typically, however, one will use an enhancer from a eukaryotic cell virus. Examples include the SV40 enhancer on the late side of the replication origin (bp 100-270), the cytomegalovirus early promoter enhancer (CMV), the polyoma enhancer on the late side of the replication origin, and adenovirus enhancers. See also Yaniv, Nature, 297:17-18 (1982) on enhancing elements for activation of eukaryotic promoters. The enhancer may be spliced into the vector at a position 5′ or 3′ to the product gene, but is preferably located at a site 5′ from the promoter.

The DNA construct of the present invention has a transcriptional initiation site following the transcriptional regulatory region and a transcriptional termination region following the product gene (see, e.g., FIG. 1). These sequences are provided in the DNA construct using techniques which are well known in the art.

The DNA construct normally forms part of an expression vector which may have other components such as an origin of replication (i.e., a nucleic acid sequence that enables the vector to replicate in one or more selected host cells) and, if desired, one or more additional selectable gene(s). Construction of suitable vectors containing the desired coding and control sequences employs standard ligation techniques. Isolated plasmids or DNA fragments are cleaved, tailored, and religated in the form desired to generate the plasmids required.

Generally, in cloning vectors the origin of replication is one that enables the vector to replicate independently of the host chromosomal DNA, and includes origins of replication or autonomously replicating sequences. Such sequences are well known. The 2μ plasmid origin of replication is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Most expression vectors are “shuttle” vectors, i.e., they are capable of replication in at least one class of organisms but can be transfected into another organism for expression. For example, a vector is cloned in E. coli and then the same vector is transfected into yeast or mammalian cells for expression even though it is not capable of replicating independently of the host cell chromosome.

For analysis to confirm correct sequences in plasmids constructed, plasmids from the transformants are prepared, analyzed by restriction, and/or sequenced by the method of Messing et al., Nucleic Acids Res., 9:309 (1981) or by the method of Maxam et al., Methods in Enzymology, 65:499 (1980).

The expression vector having the DNA construct prepared as discussed above is transformed into a eukaryotic host cell. Suitable host cells for cloning or expressing the vectors herein are yeast or higher eukaryote cells.

Eukaryotic microbes such as filamentous fungi or yeast are suitable hosts for vectors containing the product gene. Saccharomyces cerevisiae, or common baker's yeast, is the most commonly used among lower eukaryotic host microorganisms. However, a number of other genera, species, and strains are commonly available and useful herein, such as S. pombe (Beach and Nurse, Nature, 290:140 (1981)), Kluyveromyces lactis (Louvencourt et al., J. Bacteriol., 737 (1983)), kyarrowia (EP 402,226), Pichia pastoris (EP 183,070), Trichoderma reesia (EP 244,234), Neurospora crassa (Case et al., Proc. Natl. Acad. Sci. USA, 76:5259-5263 (1979)), and Aspergillus hosts such as A. nidulans (Ballance et al., Biochem. Biophys. Res. Commun., 112:284-289 (1983); Tilburn et al., Gene, 26:205-221 (1983); Yelton et al., Proc. Natl. Acad. Sci. USA, 81:1470-1474 (1984)) and A. niger (Kelly and Hynes, EMBO J., 4:475-479 (1985)).

Suitable host cells for the expression of the product gene are derived from multicellular organisms. Such host cells are capable of complex processing and glycosylation activities. In principle, any higher eukaryotic cell culture is workable, whether from vertebrate or invertebrate culture. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosphila melanogaster (fruitfly), and Bombyx mori host cells have been identified. See, e.g., Luckow et al., Bio/Technology, 6:47-55 (1988); Miller et al., in Genetic Engineering, Setlow, J. K. et al., eds., Vol. 8 (Plenum Publishing, 1986), pp. 277-279; and Maeda et al., Nature, 315:592-594 (1985). A variety of such viral strains are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can be utilized as hosts. Typically, plant cells are transfected by incubation with certain strains of the bacterium Agrobacterium tumefaciens, which has been previously manipulated to contain the product gene. During incubation of the plant cell culture with A. tumefaciens, the product gene is transferred to the plant cell host such that it is transfected, and will, under appropriate conditions, express the product gene. In addition, regulatory and signal sequences compatible with plant cells are available, such as the nopaline synthase promoter and polyadenylation signal sequences. Depicker et al., J. Mol. Appl. Gen., 1:561 (1982). In addition,. DNA segments isolated from the upstream region of the T-DNA 780 gene are capable of activating or increasing transcription levels of plant-expressible genes in recombinant DNA-containing plant tissue. EP 321,196 published 21 Jun. 1989.

However, interest has been greatest in vertebrate cells, and propagation of vertebrate cells in culture (tissue culture) has become a routine procedure in recent years (Tissue Culture, Academic Press, Kruse and Patterson, editors (1973)). Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells/-DHFR (CHO, Urlaub and Chasin, Proc. Natl. Acad. Sci. USA, 77:4216 (1980)); dp12.CHO cells (EP 307,247 published 15 Mar. 1989); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2).

Host cells are transformed with the above-described expression or cloning vectors of this invention and cultured in conventional nutrient media modified as appropriate for inducing promoters, selecting transformants, or amplifying the genes encoding the desired sequences.

Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) may be used. General aspects of mammalian cell host system transformations have been described by Axel in U.S. Pat. No. 4,399,216 issued 16 Aug. 1983. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells such as by nuclear injection or by protoplast fusion may also be used.

In preferred embodiments the DNA is introduced into the host cells using electroporation, lipofection or polyfection techniques. In a particularly preferred embodiment, the transfection is performed in a spinner vessel as illustrated by Example 3 or in some other form of suspension culture. Transfection performed in a spinner vessel is also referred to as “spinner transfection”. Culturing the cells in suspension allows them to reach a cell density of at least about 5×10⁵/ml and more preferrably at least about 1.5×10⁶/ml prior to transfection. See Andreason, J. Tiss. Cult. Meth., 15:56-62 (1993), for a review of electroporation techniques useful for practicing the claimed invention. It was discovered that these techniques for introducing the DNA construct into the host cells are preferable over calcium phosphate precipitation techniques insofar as the latter could cause the DNA to break up and form concatemers.

The mammalian host cells used to express the product gene herein may be cultured in a variety of media as discussed in the definitions section above. The media is formulated to provide selective nutrient conditions or a selection agent to select transformed host cells which have taken up the DNA construct (either as an intra- or extra-chromosomal element). To achieve selection of the transformed eukaryotic cells, the host cells may be grown in cell culture plates and individual colonies expressing one or both of the selectable genes (and thus the product gene) can be isolated and grown in growth medium under defined conditions. The host cells are then analyzed for transcription and/or transformation as discussed below. The culture conditions, such as temperature, pH, and the like, are those previously used with the host cell selected for expression, and will be apparent to the ordinarily skilled artisan.

Gene amplification and/or expression may be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA or mRNA analysis), or in situ hybridization, using an appropriately labeled probe, based on the sequences provided herein. Various labels may be employed, most commonly radioisotopes, particularly ³²P. However, other techniques may also be employed, such as using biotin-modified nucleotides for introduction into a polynucleotide. The biotin then serves as the site for binding to avidin or antibodies, which may be labeled with a wide variety of labels, such as radionuclides, fluorescence, enzymes, or the like. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of tissue sections and assay of cell culture or body fluids, to quantitate directly the expression of gene product. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. A particularly sensitive staining technique suitable for use in the present invention is described by Hsu et al., Am. J. Clin. Path., 75:734-738 (1980).

In the preferred embodiment protein expression is measured using ELISA as described in Example 1 herein.

The product of interest preferably is recovered from the culture medium as a secreted polypeptide, although it also may be recovered from host cell lysates when directly expressed without a secretory signal. When the product gene is expressed in a recombinant cell other than one of human origin, the product of interest is completely free of proteins or polypeptides of human origin. However, it is necessary to purify the product of interest from recombinant cell proteins or polypeptides to obtain preparations that are substantially homogeneous as to the product of interest. As a first step, the culture medium or lysate is centrifuged to remove particulate cell debris. The product of interest thereafter is purified from contaminant soluble proteins and polypeptides, for example, by fractionation on immunoaffinity or ion-exchange columns; ethanol precipitation; reverse phase HPLC; chromatography on silica or on a cation exchange resin such as DEAE; chromatofocusing; SDS-PAGE; ammonium sulfate precipitation; gel electrophoresis using, for example, Sephadex G-75; chromatography on plasminogen columns to bind the product of interest and protein A Sepharose columns to remove contaminants such as IgG.

The following examples are offered by way of illustration only and are not intended to limit the invention in any manner. All patent and literature references cited herein are expressly incorporated by reference.

### EXAMPLE 1

### 2C4 Production Using the Fusion Construct Expression Vector

Vectors related to those described by Lucas et al (Lucas B K, Giere L M, DeMarco R A, Shen A, Chisholm V and Crowley C. High-level production of recombinant proteins in CHO cells using a dicistronic DHFR intron expression vector. (1996) Nucleic Acids Res. 24(9), 1774-1779.), which contain an intron between the SV40 promoter and enhancer and the cDNA that encodes the polypeptide of interest, were constructed. The intron is boardered on its 3′ and 5′ ends, respectively, by a splice donor site derived from cytomegalovirus immediate early gene (CMVIE), and a splice acceptor site from an IgG heavy chain variable region (V_{H}) gene (Eaton et al., Biochem., 25:8343 (1986)). The splice sites selected provide slightly inefficient splicing such that only about 90% of the transcripts produced are intron free. Previous studies have demonstrated that when a selectable marker such as DHFR is integrated within this intron,as in the plasmid pSV.ID, marker gene transcription proceeds from any unspliced transcripts, providing a highly efficient means of maintaining linkage between the expression of the marker gene and the cDNA of interest as well as enhanced product expression relative to expression of the marker gene.

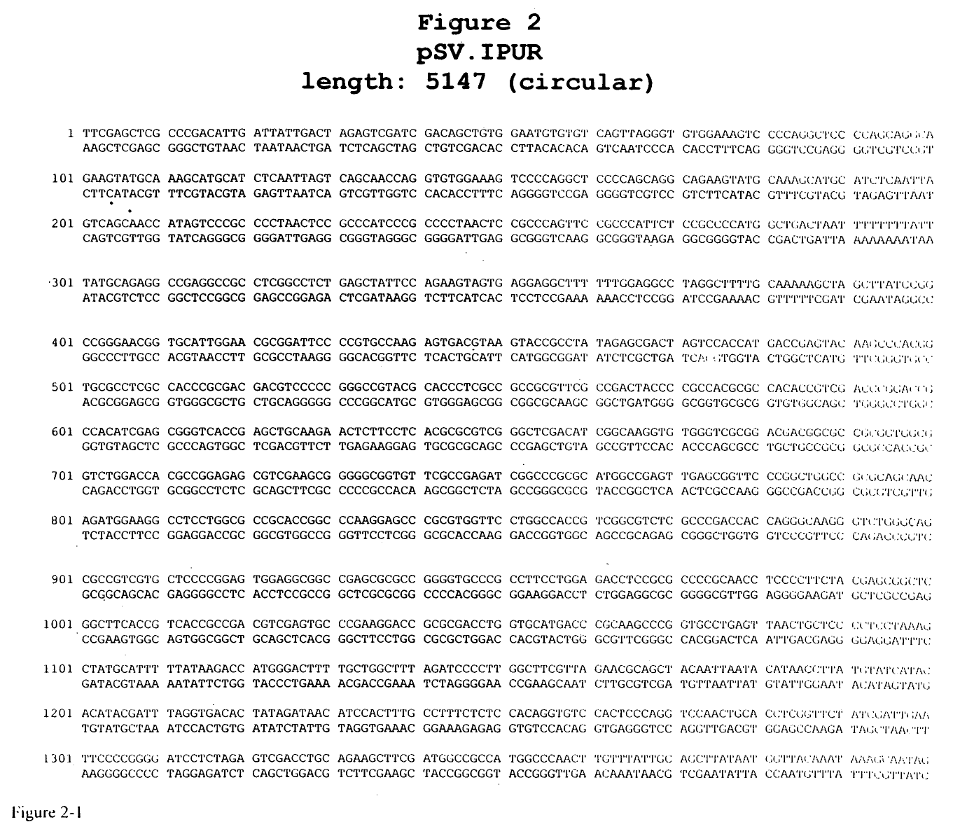

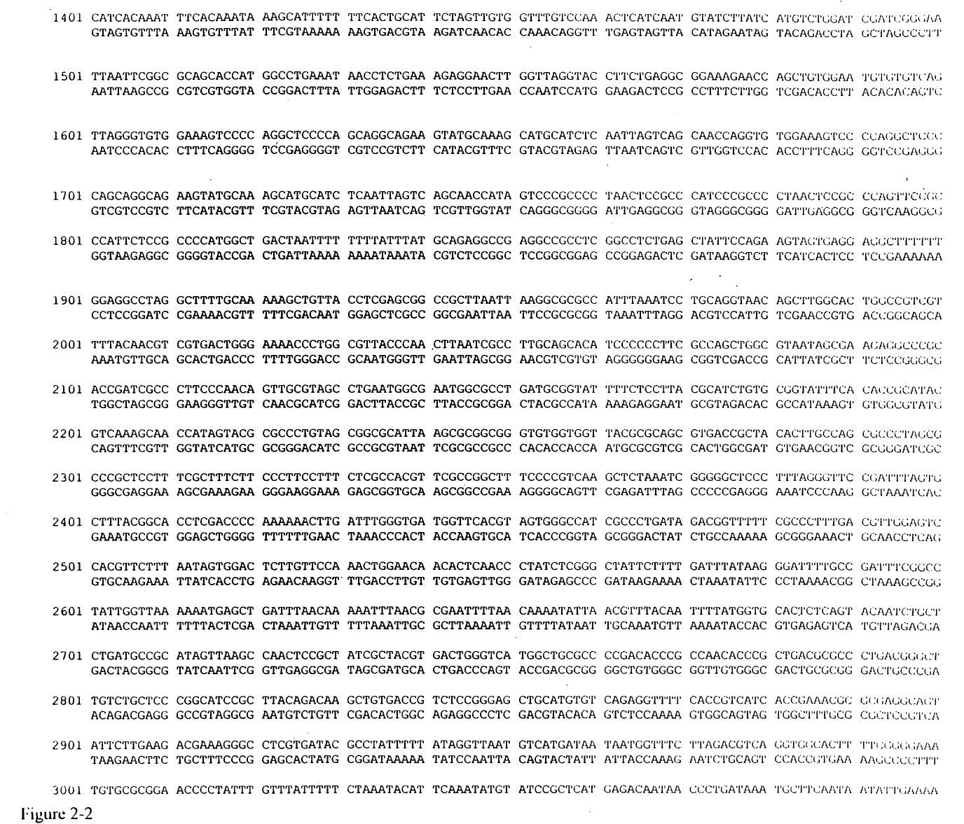

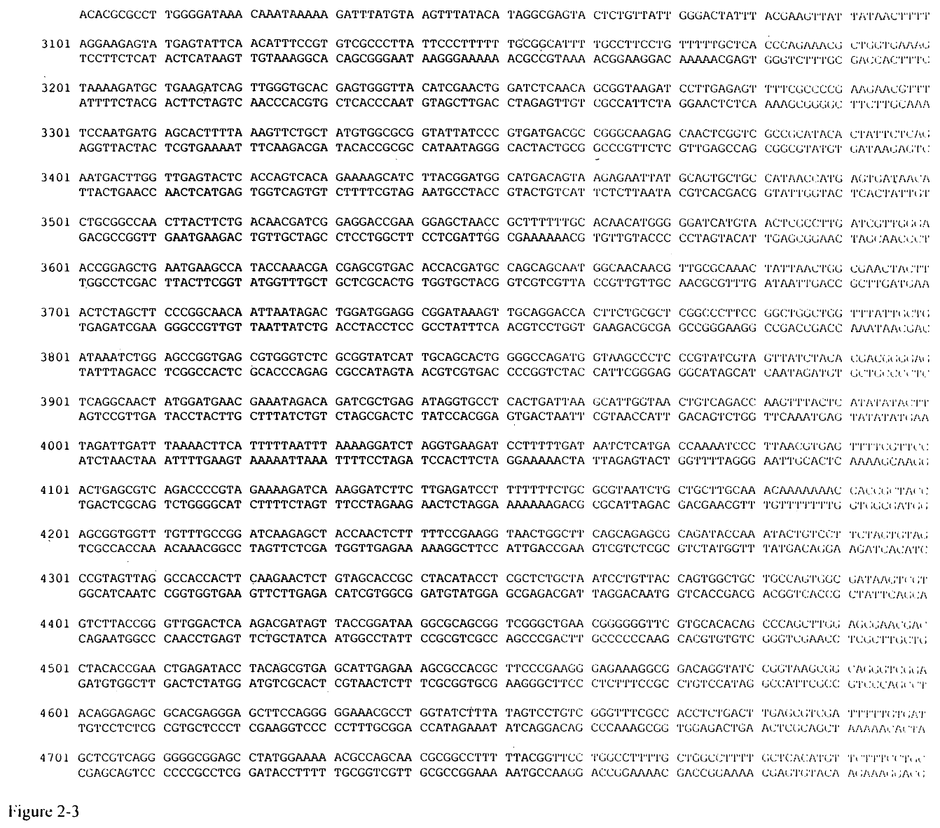

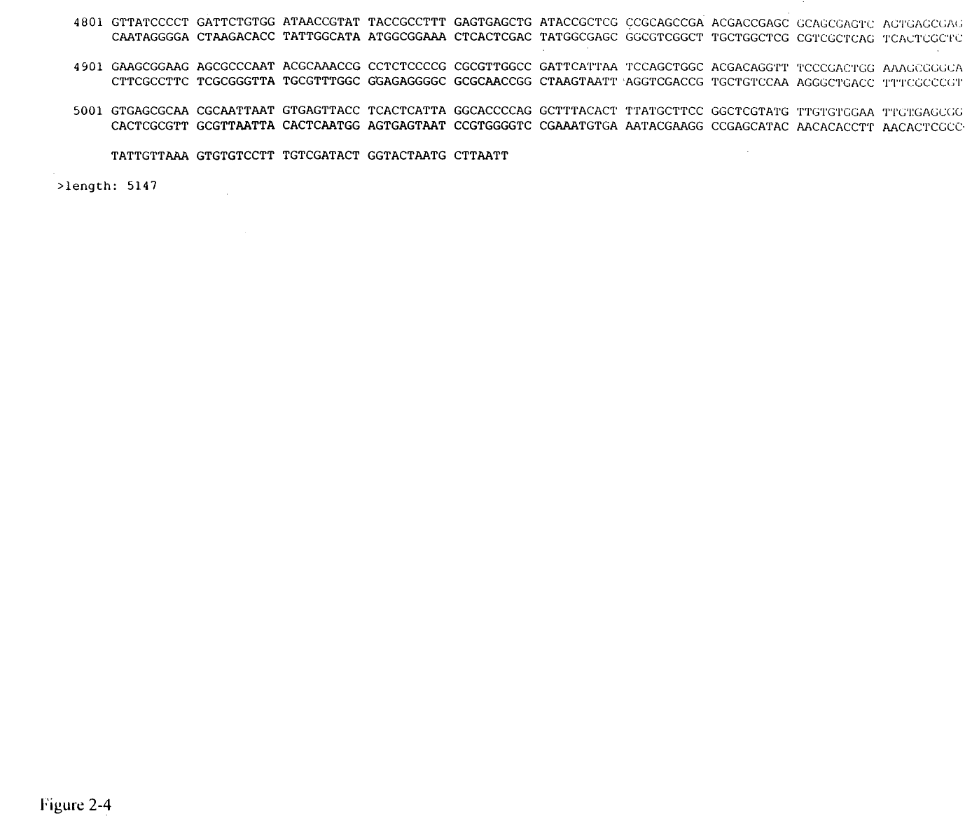

Vectors containing a murine puromycin/DHFR fusion sequence in the intron following the SV40 promoter elements were constructed by linearizing a pSV.IPUR plasmid, which contained the puromycin resistance gene in an intron following the SV40 promoter/enhancer (pSV.IPUR, FIGS. 1 and 2), with Hpa I immediately following the end of the puromycin ORF. A 564 bp PCR fragment containing the entire coding region for the murine DHFR gene was subsequently ligated into this linearized vector 3′ of the puromycin resistance gene. The stop codon TAG between the puromycin resistance gene and the DHFR gene was deleted by site-directed mutagenesis resulting in a pSV.I plasmid containing a Puro/DHFR fusion gene within the intron of the expression cassette (pSV.IPD, FIGS. 1 and 4).

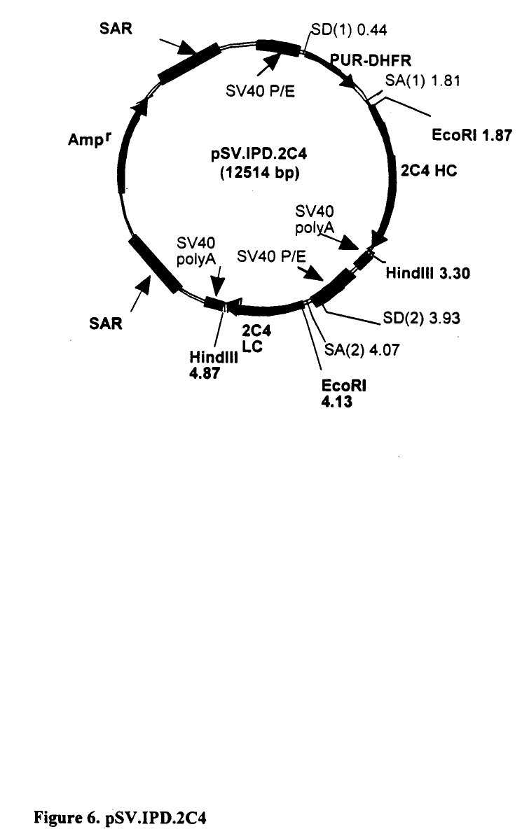

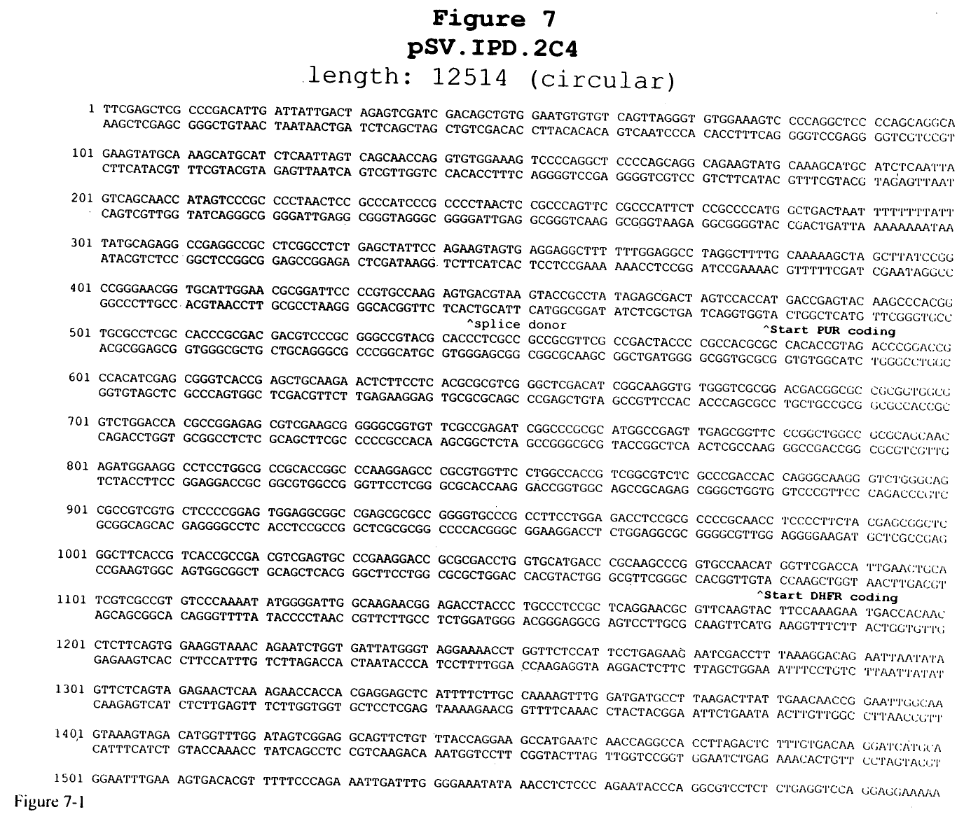

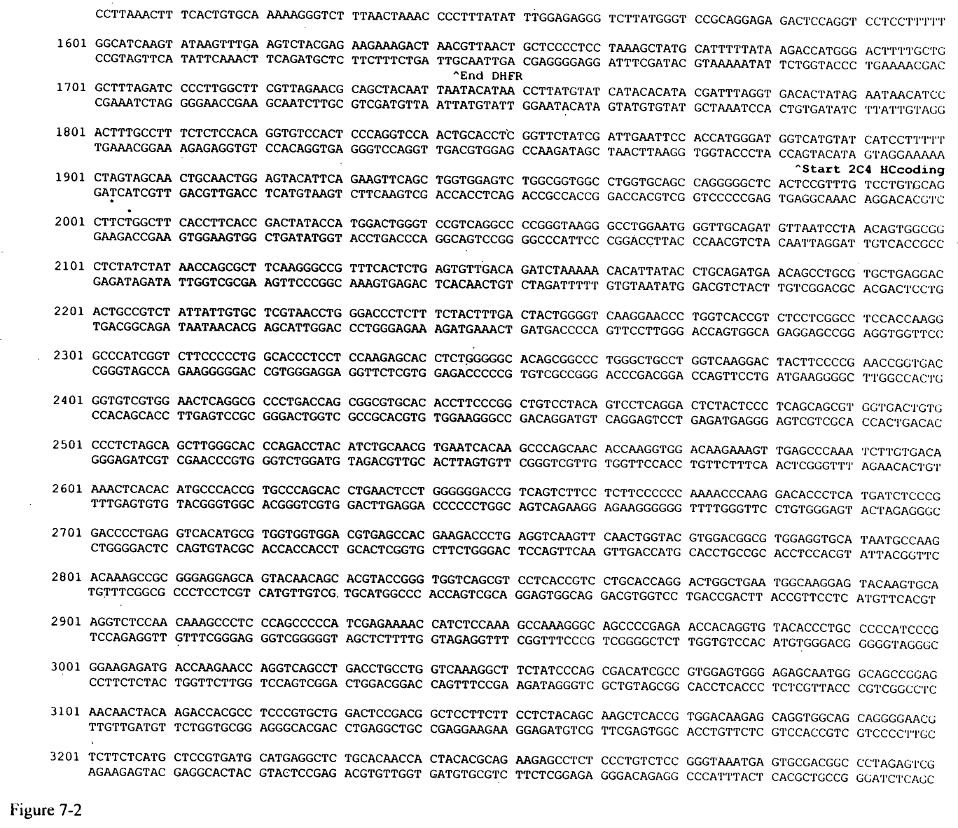

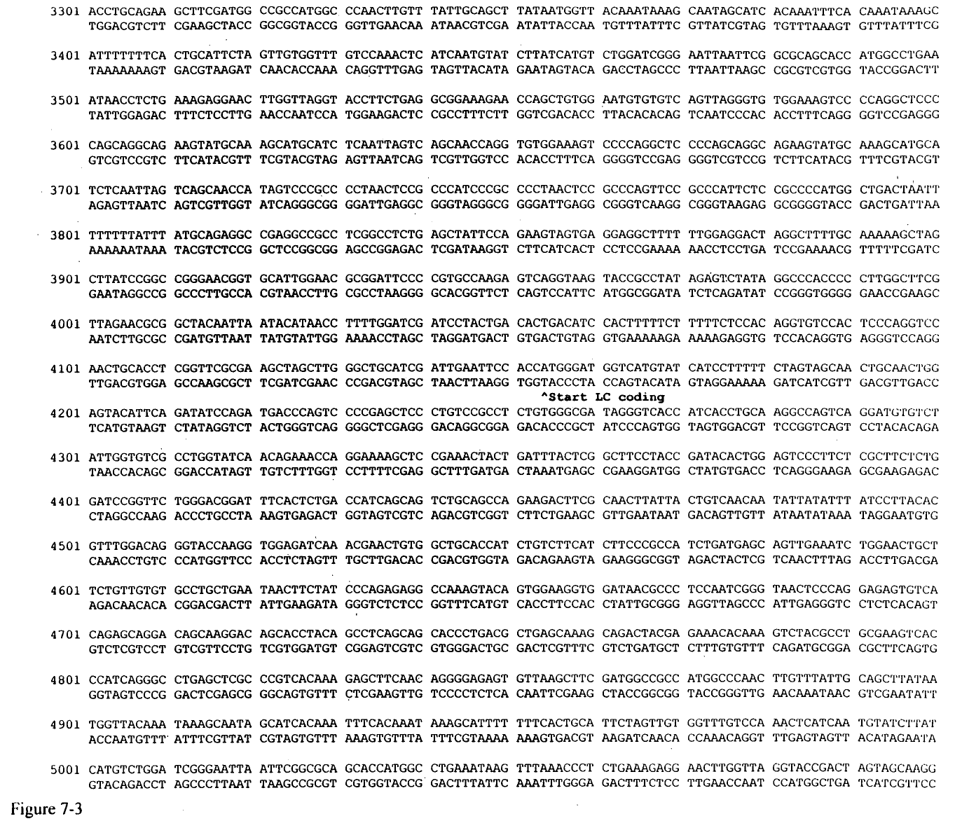

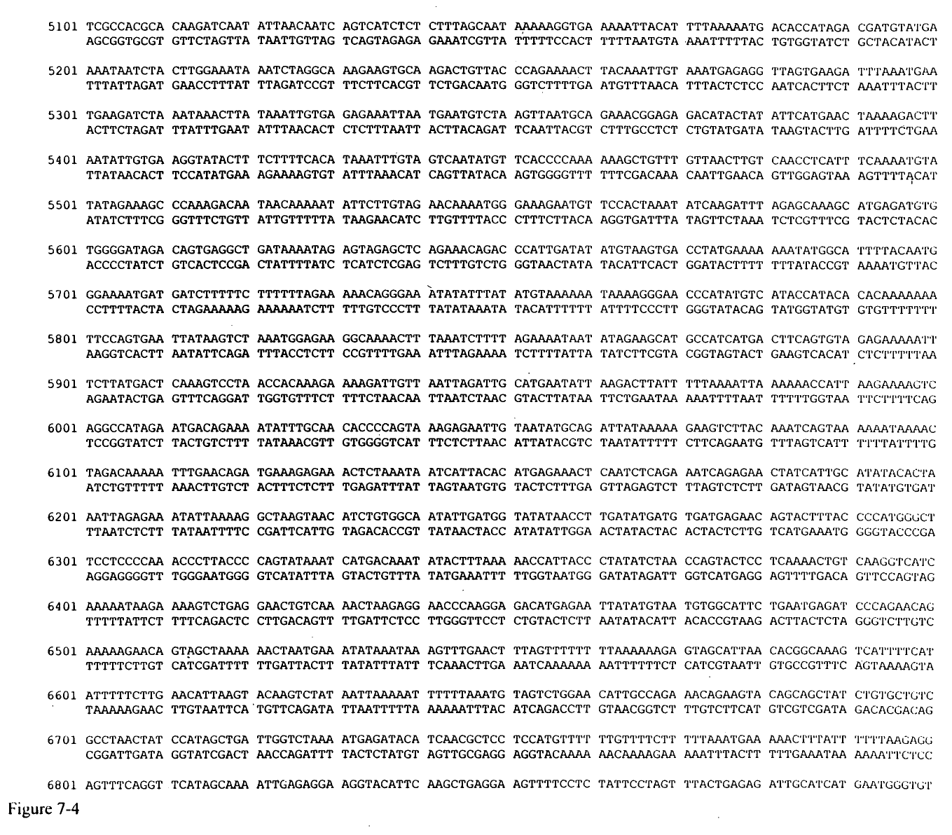

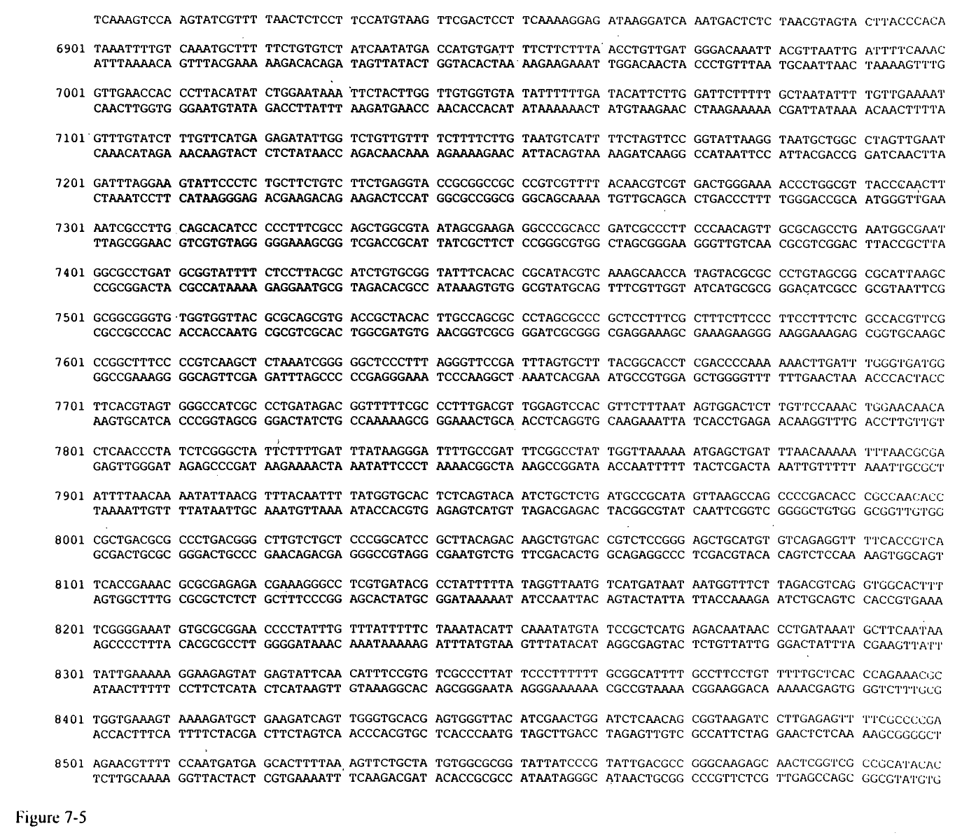

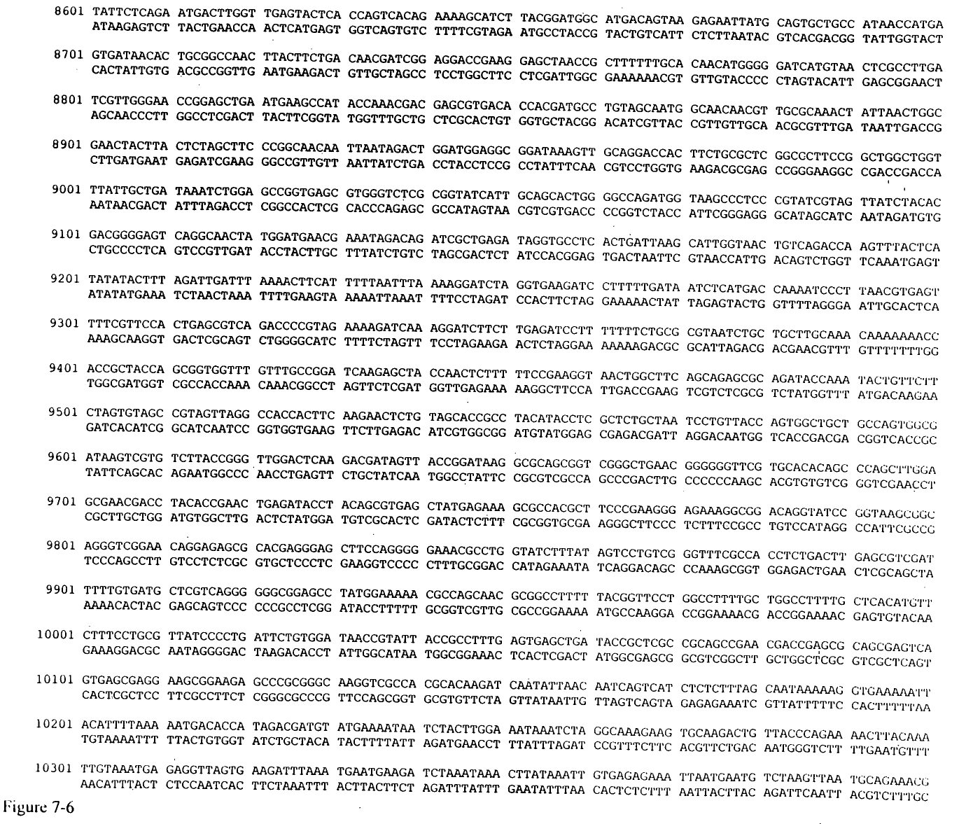

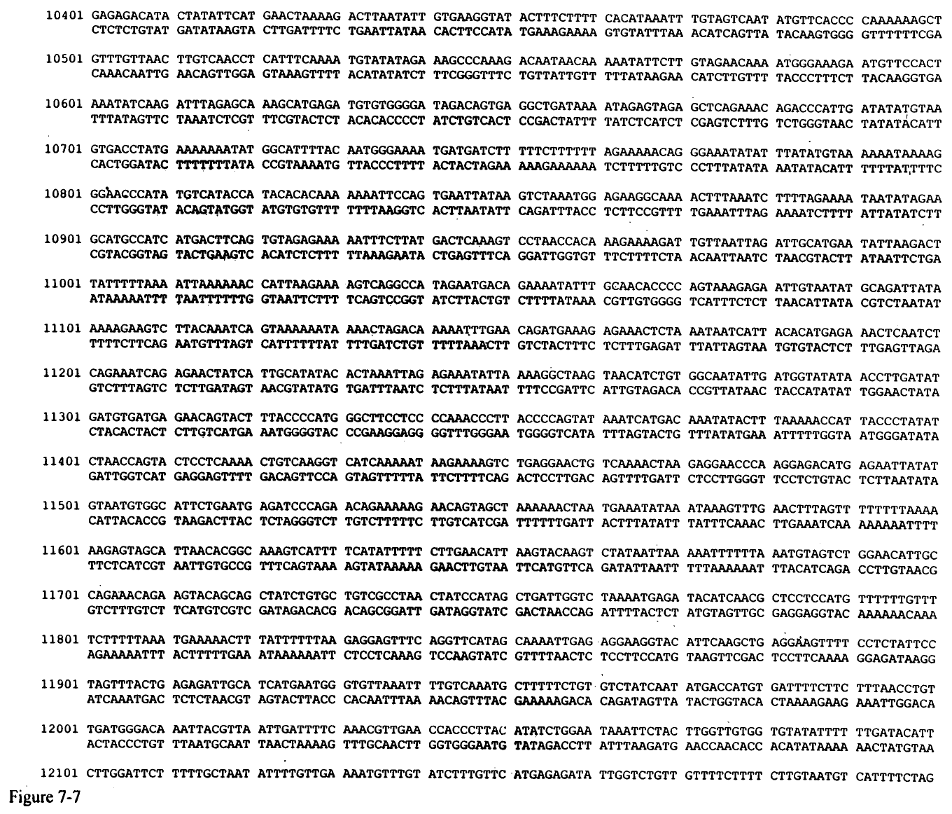

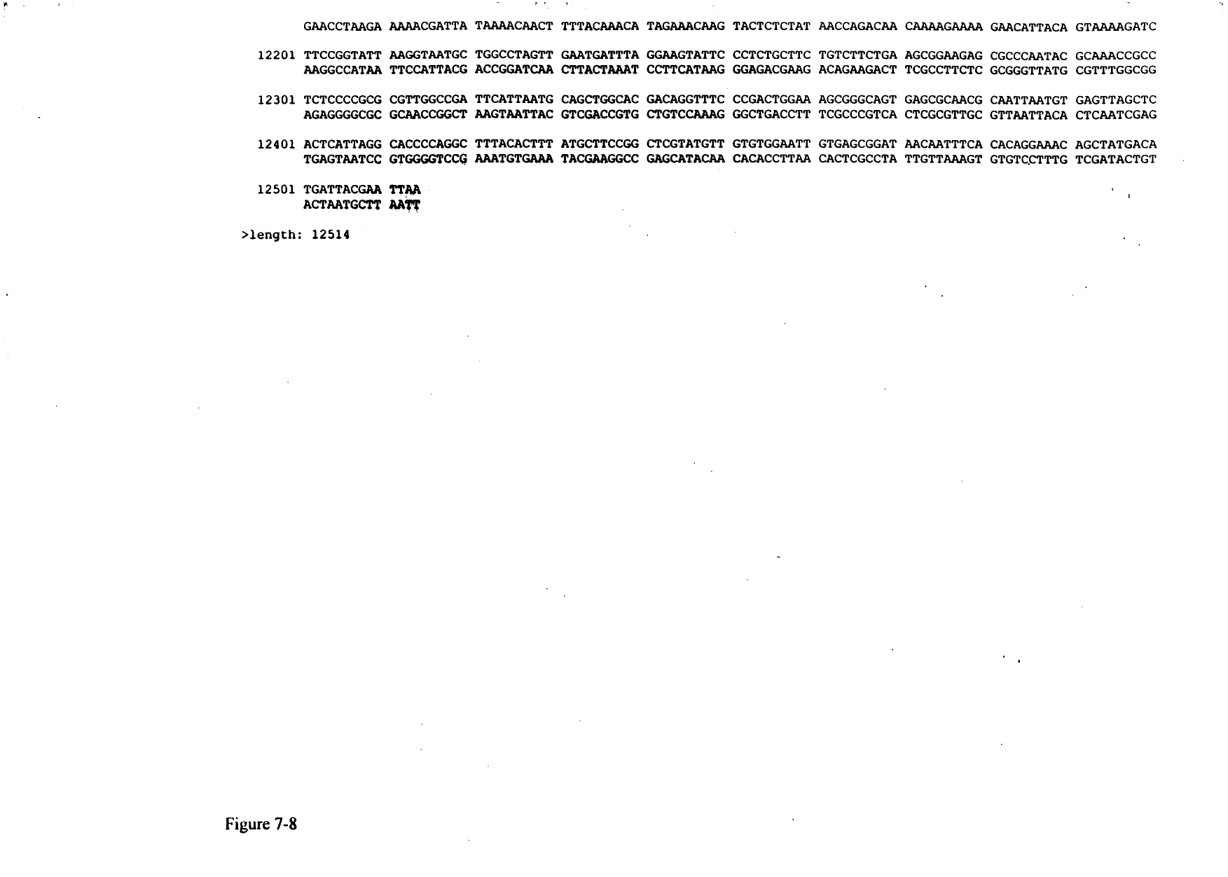

The cDNA of the Heavy chain (HC) and light chain (LC) sequences of an anti-HER2 Neu antibody, 2C4, were inserted into pSV.IPD as shown in FIG. 6. The sequence of the resulting pSV.IPD.2C4 vector is shown in FIG. 7. Data collected using the pSV.IPD.2C4 vector are shown in Table 2.

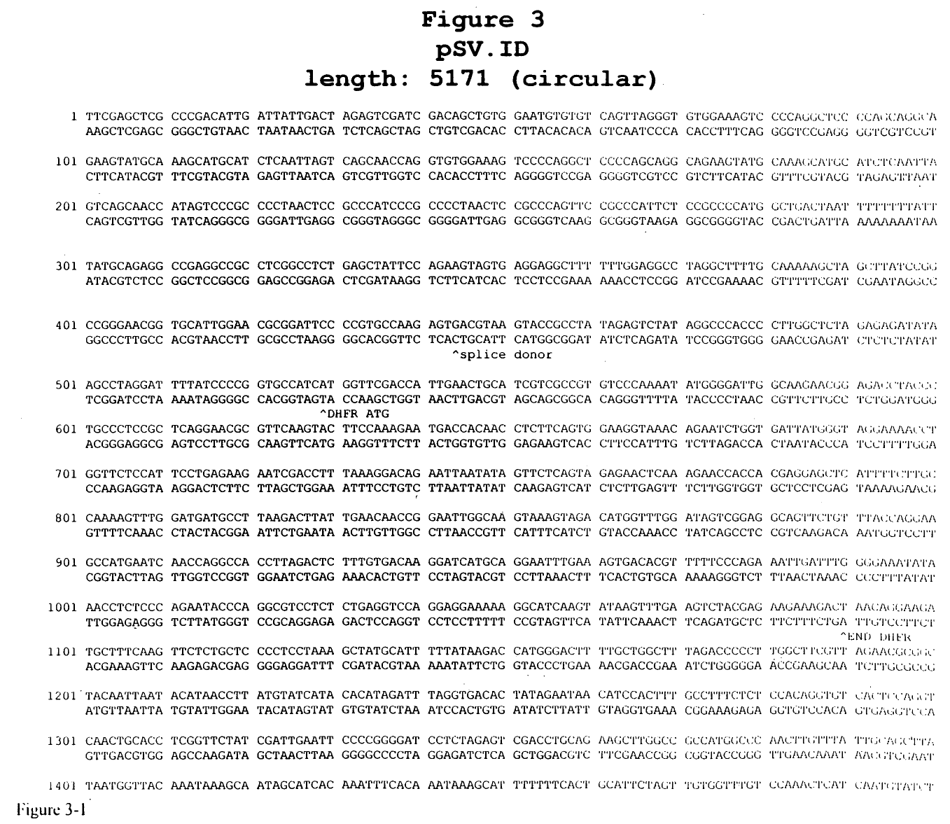

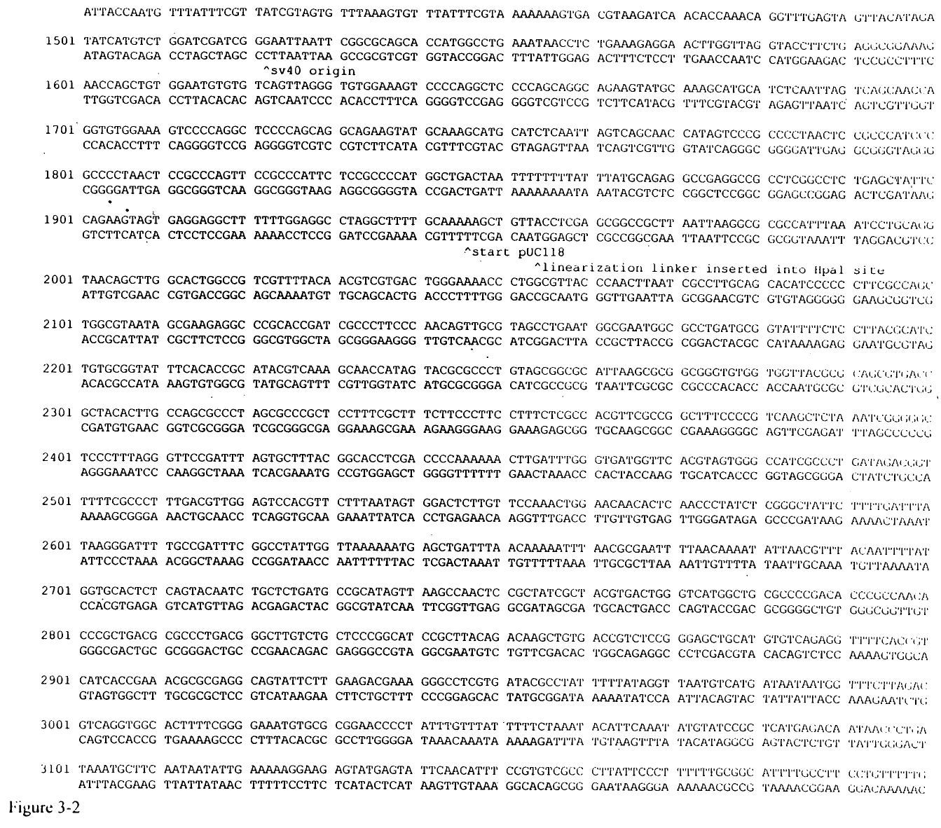

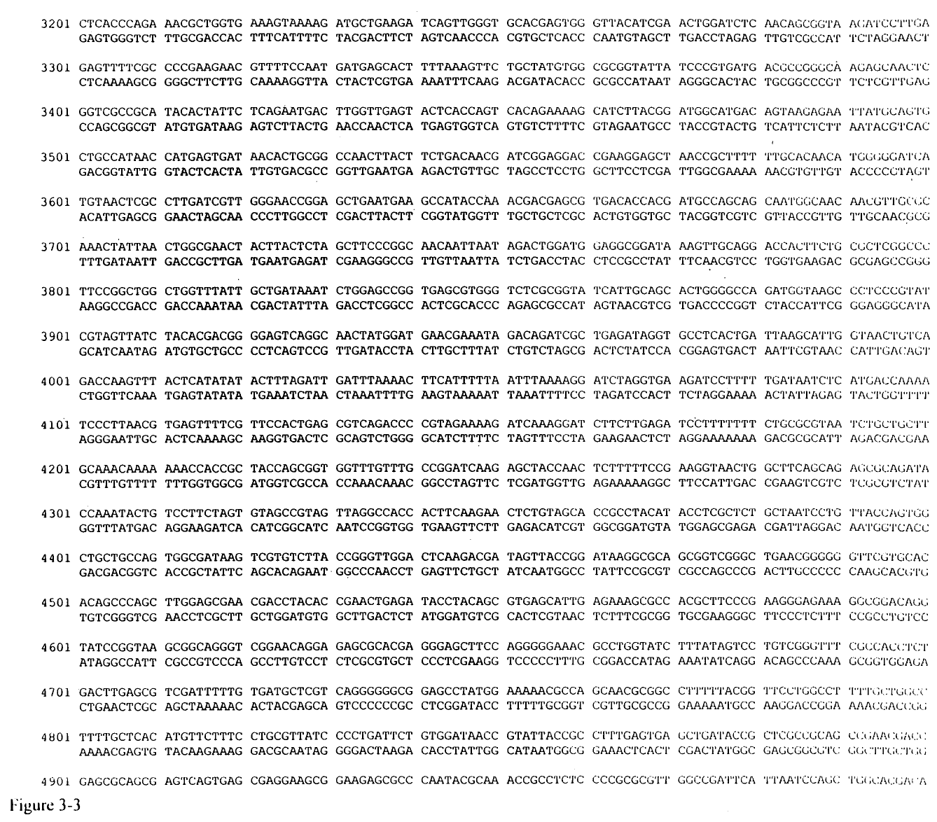

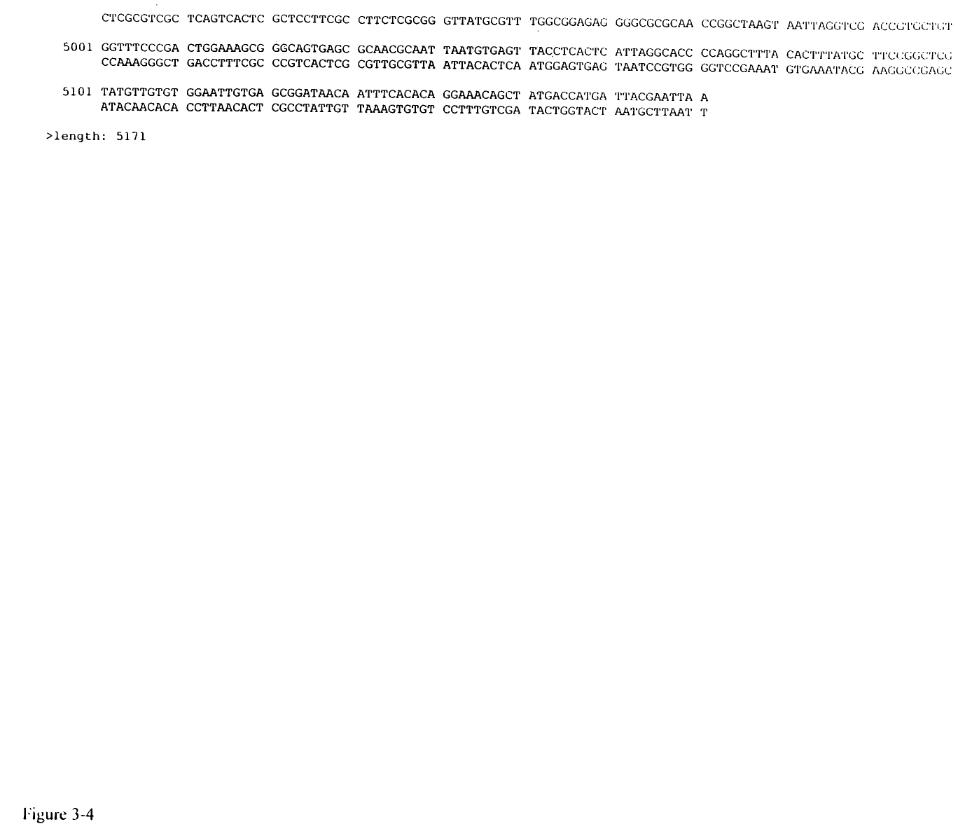

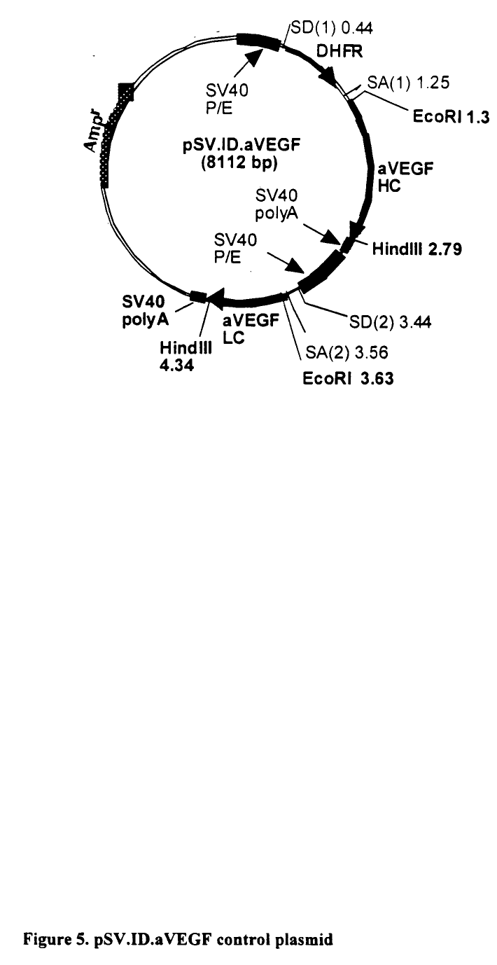

Additionally, a vector containing only a murine DHFR sequence within the intron (pSV.ID) was prepared. The DNA sequence for the pSV.ID vector is shown in FIG. 3. The preparation of such vectors is disclosed in U.S. Pat. No. 5,561,053, which is herein incorporated by reference. Into that vector, the HC and LC sequences of monoclonal antibodies to VEGF were inserted. The sequence of the resulting pSV.IPD.VEGF vector is shown in FIG. 5.

Plasmid DNA's that contained either the Puro/DHFR fusion sequences in the intron or murine DHFR alone preceding cDNA sequences for HC and LC of 2C4 and anti-VEGF, respectively were introduced into CHO DHFR minus cells by lipofection. Briefly, for transfection, 4 million CHO DUX-B11 (DHFR minus) were seeded in 10 cm plates the day before transfection. On the day of transfection, 4 ug DNA was mixed with 300 ul of serum free medium and 25 ul of polyfect from Qiagen. The mixture was incubated at room temperature for 5 to 10 minutes and added to the cells. Cells were fed with fresh glycine, hypoxanthine and thymidine-free (GHT-free) medium and twenty-four hours later, were trypsinzed and selected in fresh GHT-free medium with 0-5 nM of methotrexate (MTX) in order to select for stable DHFR+ clones. Approximately 300-400 individual clones were selected in this first round of screening for measurement of protein expression levels. Clones from each vector which expressed the highest levels of antibody were then re-exposed to higher levels of methotrexate to affect a second round of gene amplification and selection. The screening process was repeated on all available clones, the highest of which were exposed to a third round of amplification. The methotrexate concentrations used during amplification using the pSV.ID-derived vector was 50 to 1000 nM in the 2^{nd} round and 200 to 1000 nM in the 3^{rd} round. These concentrations are typically required to achieve growth-limiting toxicity, which is required to achieve sufficient selective pressure for gene amplification. Concentrations required to reach this same degree of toxicity using the pSV.IPD-derived vectors were remarkably lower.

The level of antibody expression was determined by seeding cells in 1 ml of serum-free F12:DMEM-based media supplemented with protein hydrolysate and amino acids in 24 well dishes at 3×10\hat{ }6 cells/ml or in 100 ul of similar media in individual wells of a 96 well plate. Growth media was collected after 3-4 days and titers were assayed by an ELISA directed towards the intact IgG molecule. In experiments where cells were not seeded at equal cell densities, a fluorescent measure of viable cell number was performed on each well in order to normalize expression data. An Intact IgG ELISA was performed on microtiter plates which used a capture antisera directed to framework Fab residues common in both antibodies. Media samples were added to the wells followed by washing and a horseradish peroxidase labeled second antibody directed towards common framework Fc residues was used for detection.

Table 2 presents expression level distributions of clones isolated during each round of screening of anti VEGF clones, which resulted from transfection with the plasmid containing only the DHFR sequence in the intron (pSV.ID.aVEGF), and 2C4 clones that were created using the Puro/DHFR fusion sequence in the same intron (pSV.IPD.2C4). The distribution of expression levels seen in the case of anti VEGF is typical of the performance of the vector containing only the murine DHFR gene in the intron (pSV.ID). All isolates identified in the first and second rounds of screening have relatively low expression levels. In the intial selection round, no clones with expression above 5 were isolated. At least three rounds of amplification are required to identify clones capable of specific productivity greater than 50. The 2C4 clones were screened after the first exposure to methotrexate (0-2.5 nM) and the most productive of these were exposed to a second round of amplification in 10-25 nM MTX. Cells surviving this amplification were pooled and exposed to 3^{rd} round amplification prior to selection for further screening. In contrast to the pSV.ID vetor, using the pSV.IPD vector, clones with an expression level of up to 25 were identified even in the first round of screening. Clones with an expression level greater than 25 represented 95% of the population after their third round of amplification and screening.

The data from Example 1 indicates that use of the Puro/DHFR fusion protein as the selectable marker allows for faster, more efficient isolation of highly productive CHO clones using significantly lower levels of methotrexate. The data suggests that exposure to low concentrations and stepwise increments in methotrexate allow for the efficient initial selection of highly expressing clones and subsequent gene amplification. Exposure to excessively high concentrations of methotrexate or large incremental increases in exposure often does not yield increases in gene expression since cells rapidly acquire methotrexate resistance through non-gene amplification mechanisms. Importantly, the data also shows that the Puro/DHFR fusion protein provides an unexpectedly impaired activity of the DHFR gene product or an enhanced sensitivity to methotrexate, which results in a highly stringent initial selection step, and allows efficient gene amplification at concentrations of methotrexate not frequently associated with the acquisition of drug resistance through alternative mechanisms. The ability to select cells which have incorporated the plasmid either in the presence of puromycin or methotrexate, prior to initiating exposure to methotrexate also provides a means of transferring this efficient system to DHFR (positive) host cells.

For Example 1 the structure of the expressed antibody has been extensively characterized. The proteins generated from the pSV.IPD are indistinguishable from the antibody produced by the pSV.ID vector, with no apparent increase of free heavy or light chain expressed by the pool.

TABLE 2
PERCENTAGES OF pSV.IPD.2C4 CLONES ISOLATED AT VARIOUS
EXPRESSION LEVELS AFTER MTX EXPOSURE¹

| | pSV.ID.aVEGF | pSV.IPD.2C4 | pSV.ID.aVEGF | pSV.IPD.2C4 |
|---|---|---|---|---|
| Expression Level² | 1st Rd | 1st Rd | 3rd Rd | 3rd Rd |
| <1 | 71 | 16 | 0 | 0 |
| 1-5 | 29 | 67 | 0 | 0 |
| 5-10 | 0 | 14 | 2 | 3 |
| 10-25 | 0 | 3 | 15 | 4 |
| 25-50 | 0 | 0 | 35 | 21 |
| 50-100 | 0 | 0 | 46 | 61 |
| 100-150 | 0 | 0 | 2 | 3 |

¹MTX concentration for Control SD vector = 0-10 nM 1^{st} round, 50-1000 nM 2^{nd} round, 200-1000 nM, 3^{rd} round. SD-Puro/DHFR vector = 2.5 nM 1^{st} round, 25 nM 2^{nd} round, 100 nM 3^{rd round.}
²Expression levels are in mg/ml or (mg/ml)/Fluorescent Unit

This example demonstrate the general applicability of the Puro/DHFR fusion sequence for selection of highly productive recombinant cell lines following minimal exposure to MTX.

### EXAMPLE 2

### Recombinant Protein Production Using a pSV.I Construct Containing DHFR and a Fusion Gene Other than Puro

Constructs can also be produced that contain a fusion sequence of an alternative selectable marker and DHFR within an intron region as described in Example 1. For instance starting with the vector pSVID, the coding sequences for the neomycin resistance gene (Neo), hygromycin resistance gene (Hygro), glutamine synthase (GS), thymidine kinase (TK) or zeocin (Zeo) could be inserted in frame with the start site of the murine DHFR sequence contained within the intron. The stop codon of this inserted gene would then be removed using site dirtected mutagenesis according to example 1. Depending upon the phenotype of the host cell selected, cells incorporating the plasmid could then be selected using either GHT-free or MTX containing media as described in examples 1-3 or using an appropriate quantity of the alternative selective agent. Gene expression by the resulting clones could then be amplified in the presence of increased levels of methotrexate.

### EXAMPLE 3

### Direct Selection with Plasmids SV.IPD.HP and CMV.IPD.HP After Spinner Transfection

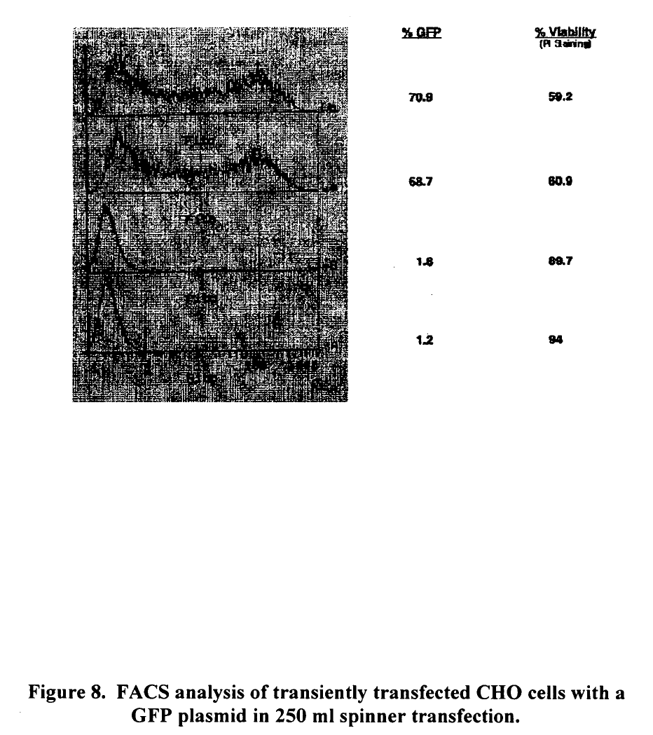

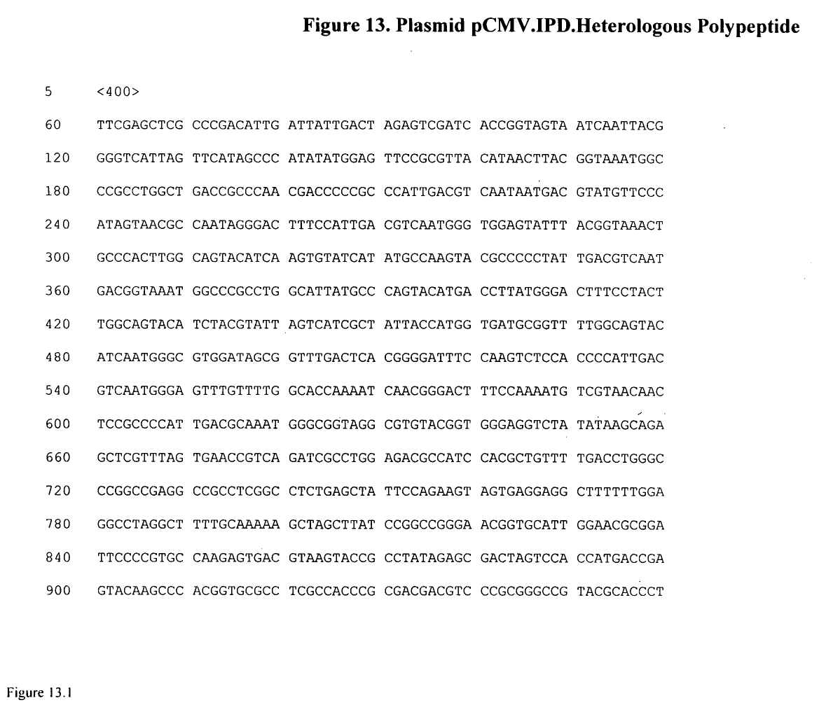

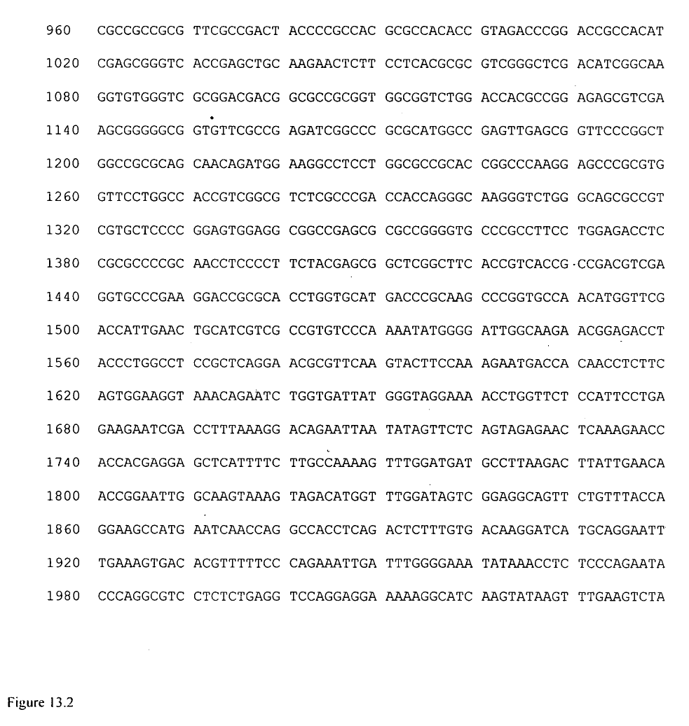

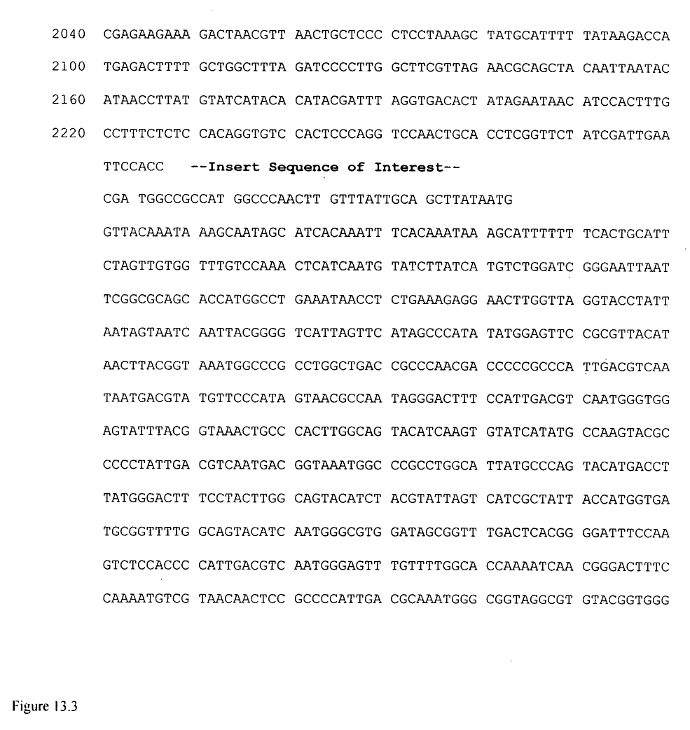

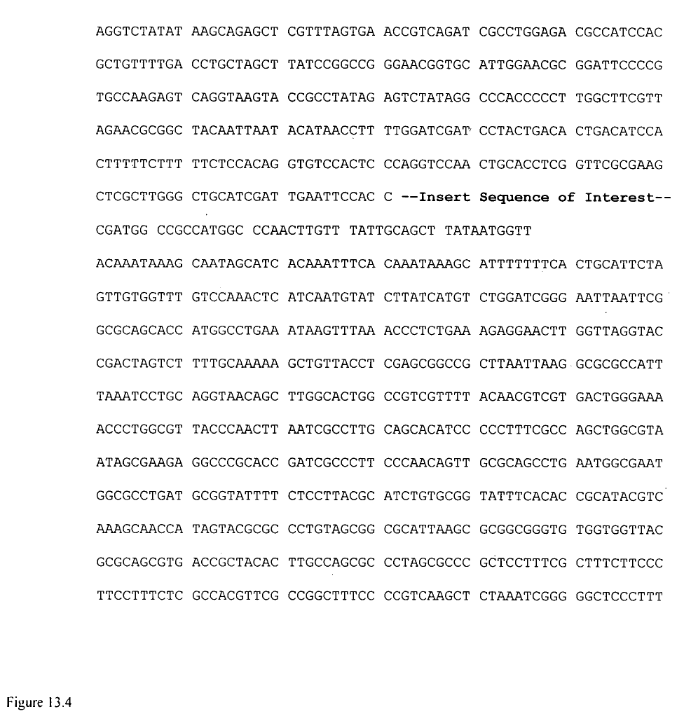

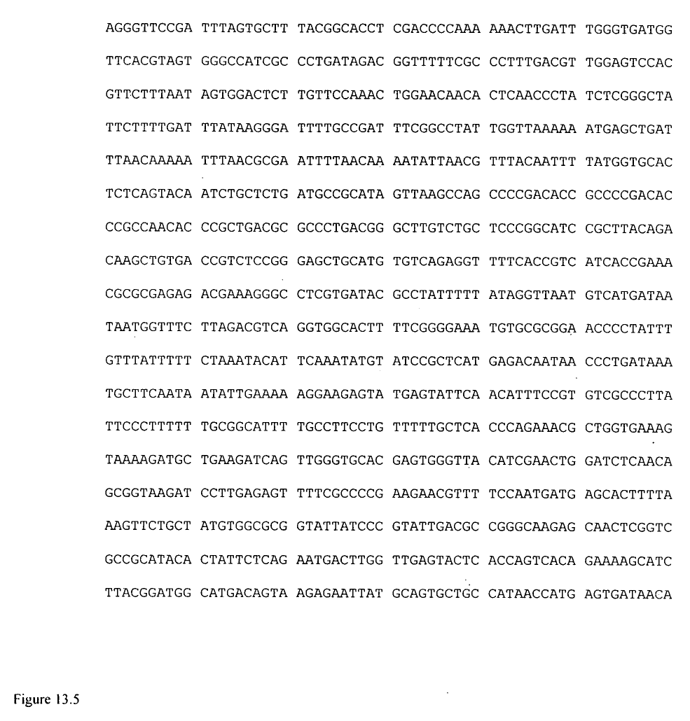

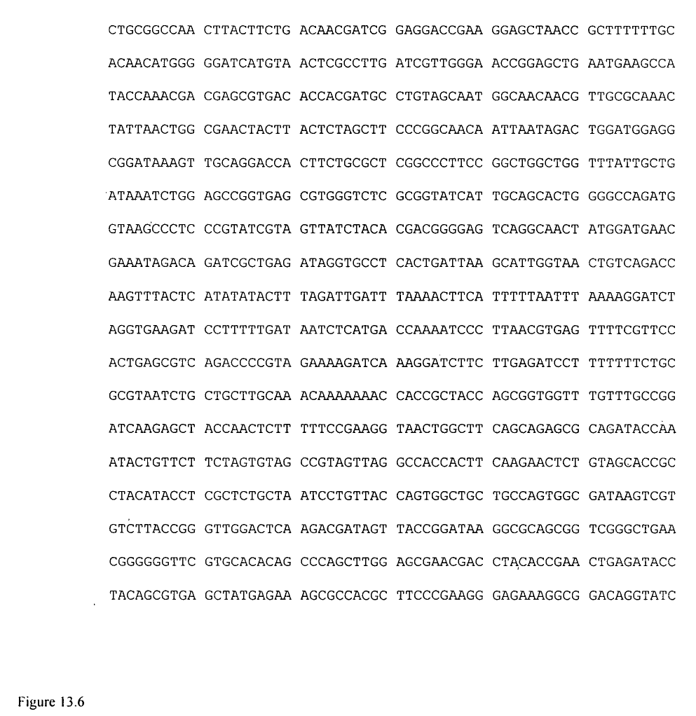

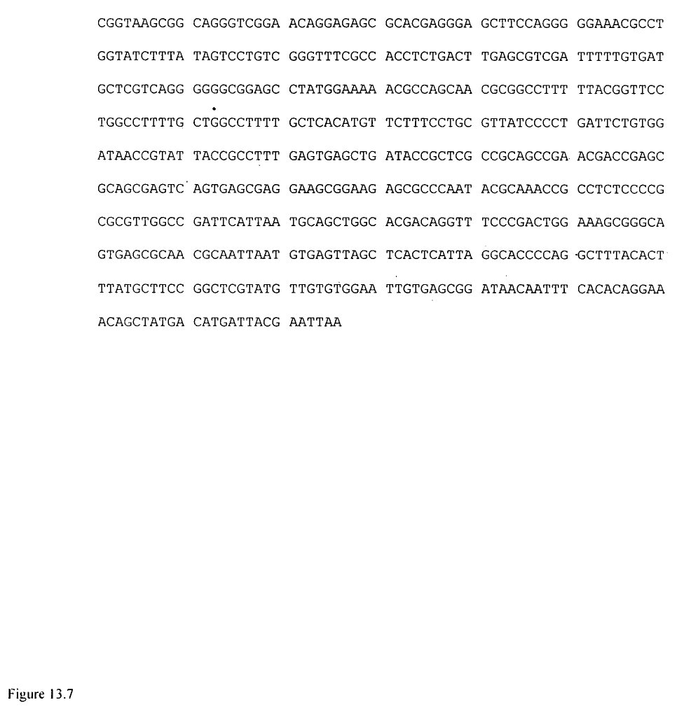

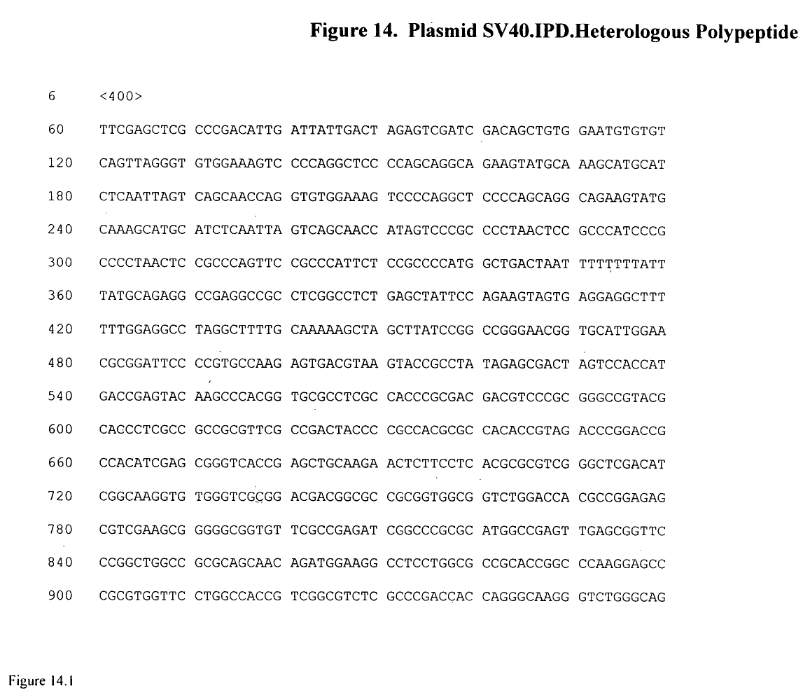

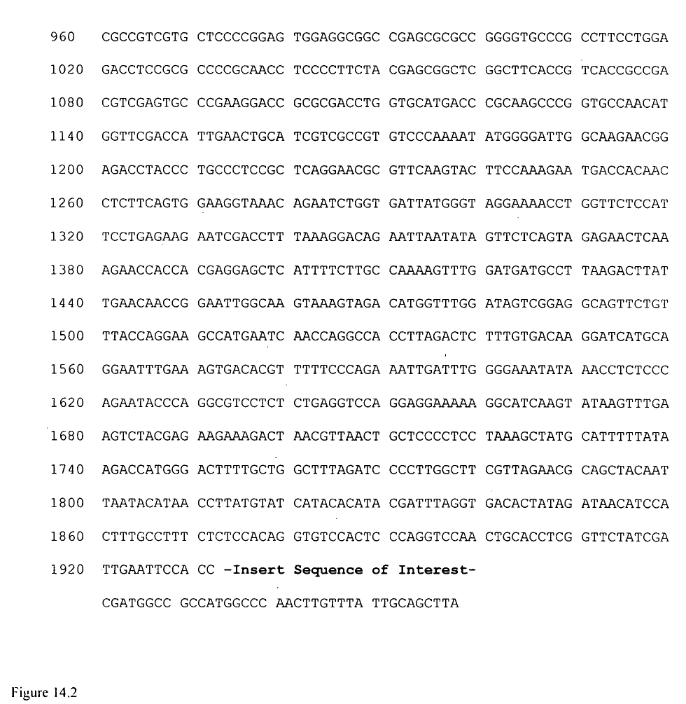

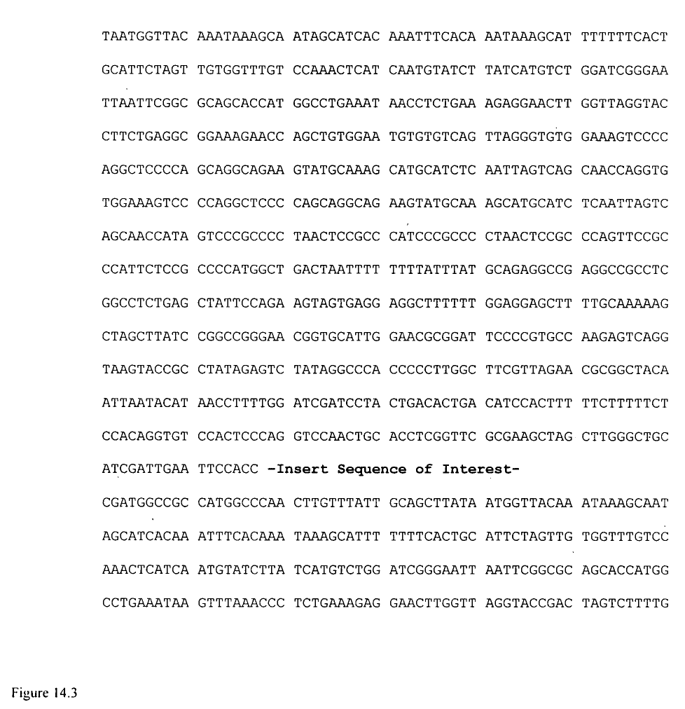

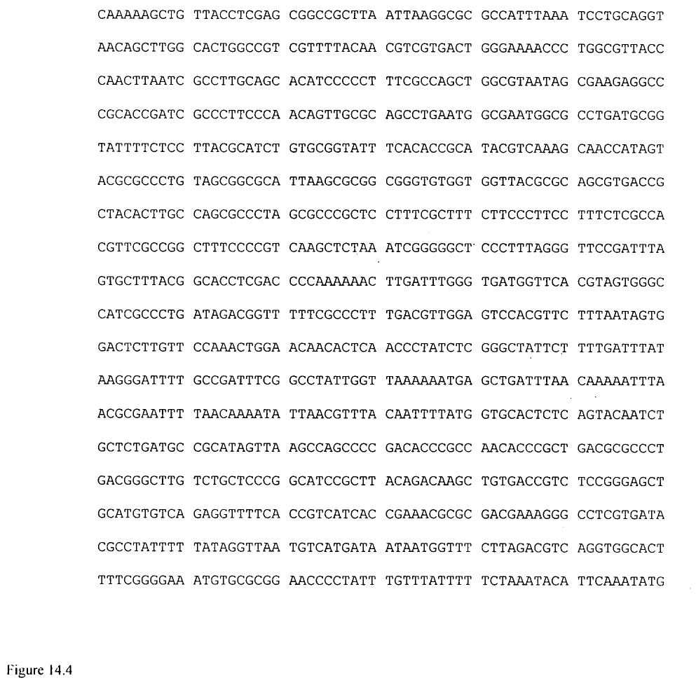

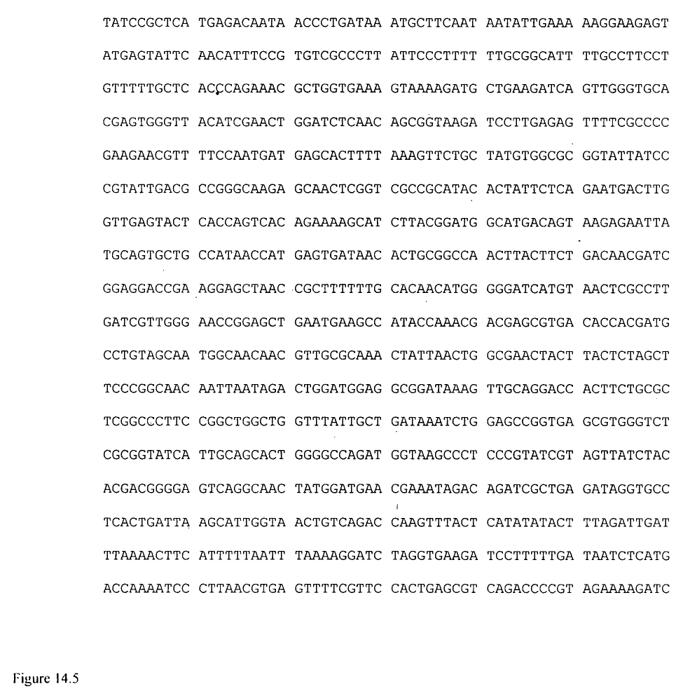

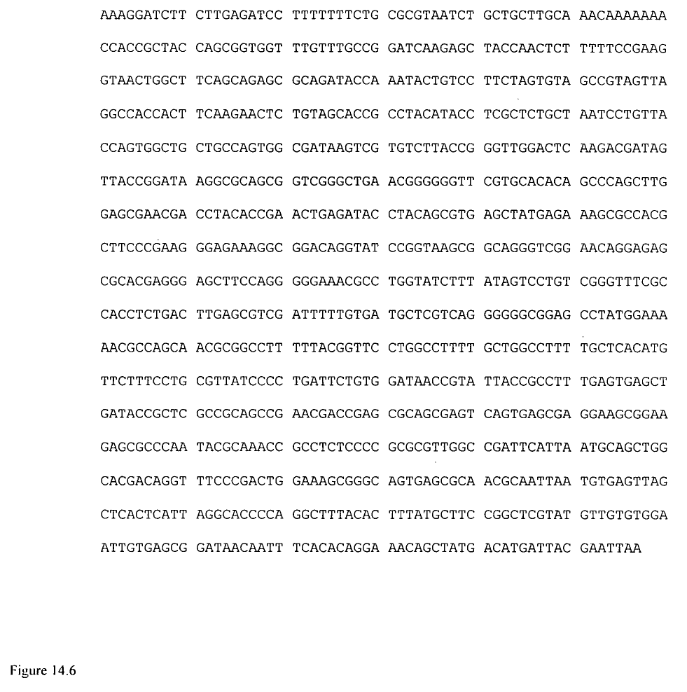

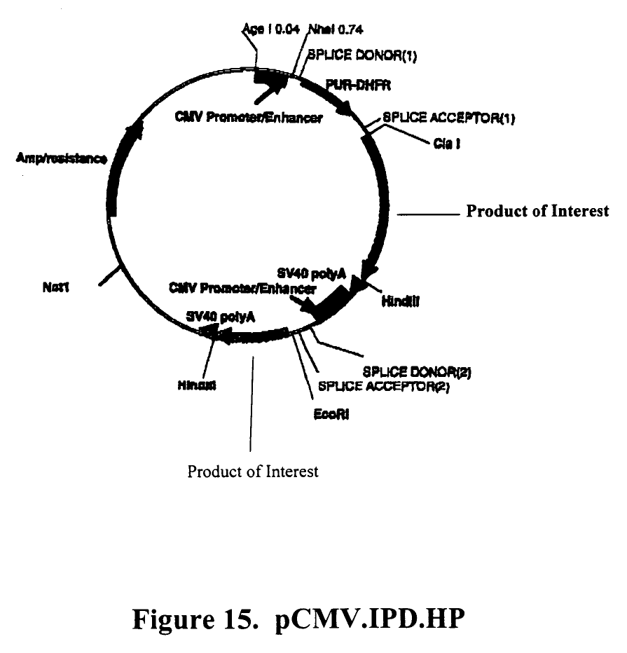

DP12 CHO cells were grown in growth medium with 5% FBS (fetal bovine serum) and 1×GHT (glycine, hypoxanthine and thymidine). The process typically took about 4 days. On day 1, cells were seeded at 4×10\hat{ }5/ml in 400 ml growth medium in a 500 ml spinner vessel and grown for 2 days at 37° C. On day 3, the exponentially grown cells were seeded at 1.5×10\hat{ }6 cells/ml in a 250 ml spinner vessel containing 200 ml of growth medium plus 5% FBS and 1×GHT. The cells were grown for 1 to 2 hours at 37° C. before transfection. During that time, serum-free growth medium and 1×GHT was warmed to 37° C. 400 μg plasmid construct DNA and 1 ml of Lipofectamine 2000® (Qiagen) were separately diluted into 25 ml of warm serum-free medium in 50 ml Falcon tubes. The solutions in the tubes were combined and incubated at room temperature for 30 minutes. The cells were then transfected with plasmid constructs pSV.IPD.HP and pCMV.IPD.HP, which constructs are illustrated in FIGS. 13 and 14, respectively. At the end of incubation, the cells were transfected by adding all 50 ml of the mixture of diluted plasmid construct and Lipofectamine 2000® to the 250 ml spinner vessel containing cells in serum-free medium, and the cells continued to grow at 37° C. for about 24 hours. On day 4, 250 ml of transfected cells were centrifuged at 1000 rpm for 5 minutes to collect the pellet. The transfection efficiency was monitored by transfecting cells with a GFP plasmid followed by FACS analysis 24 hours after transfection. The transfection efficiency with this protocol was typically approximately 55 to 70% in CHO cells as shown in FIG. 8.

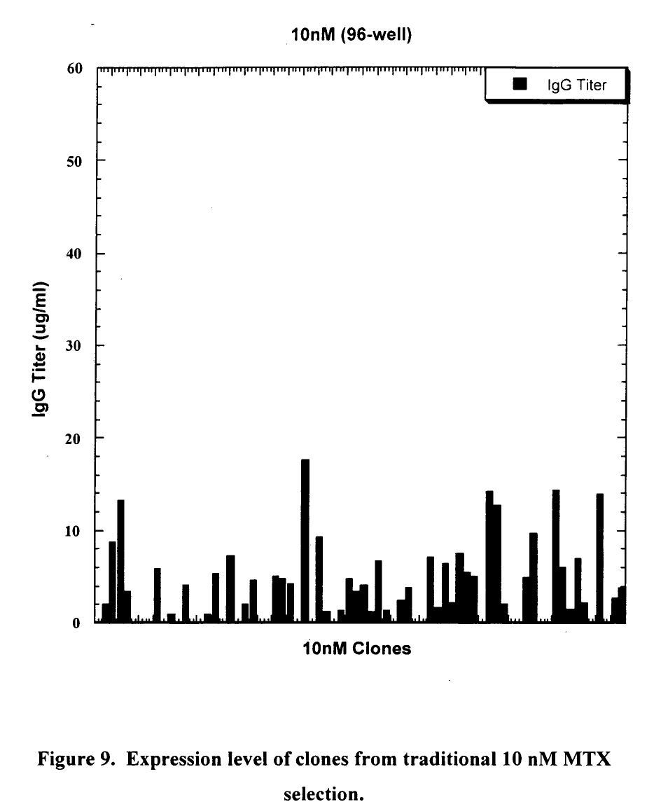

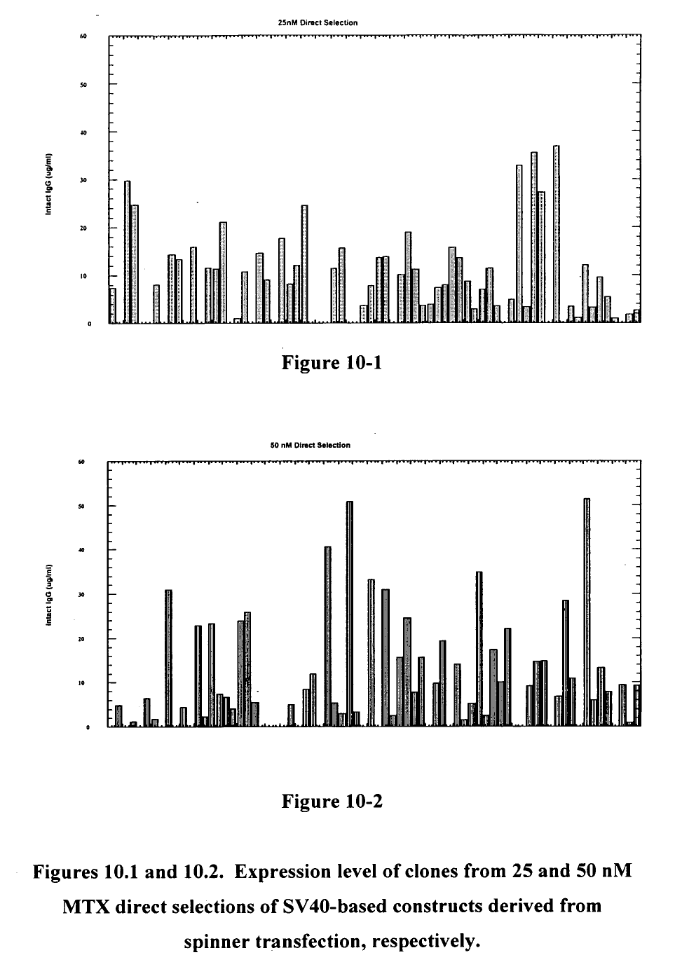

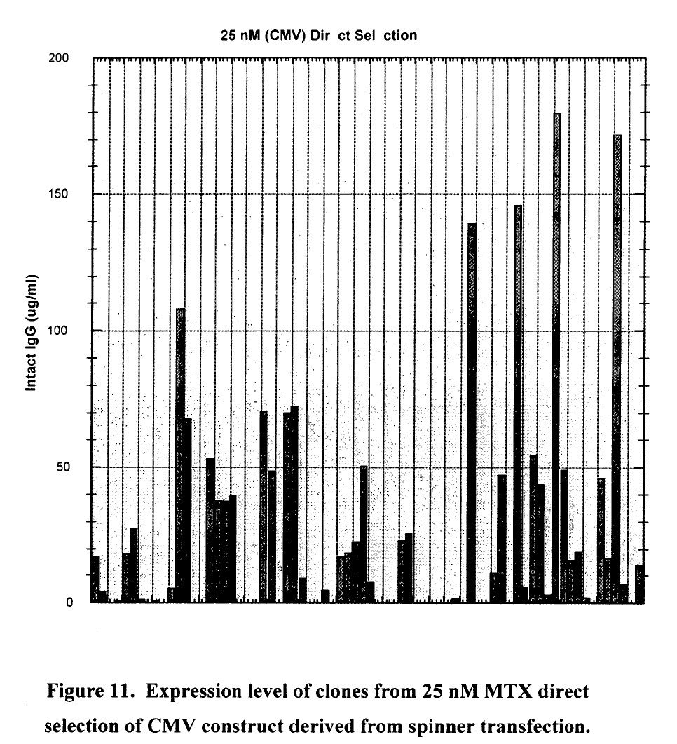

After the transfection, cells were centrifuged to collect the pellet. The pellet was then resuspended in growth medium containing methotrexate (MTX) ranging from 10 to 100 nM for either SV40 or CMV based constructs. Approximately 100 clones survived the direct selection. Cell growth medium was changed every 3 to 4 days. At approximately 2 weeks after transfection, individual clones were picked and grown in 96-well plates in growth medium containing MTX. Heterologous polypeptide expression levels were evaluated by ELISA. FIGS. 10-1, 10-2, and 11 show the results from 25 nM and 50 nM MTX selection. FIG. 9 shows heterologous polypeptide expression levels of clones from a traditional 10 nM MTX selection where the cells were not transfected in a spinner flask.

It took about 1 week for cells to grow confluent in a 96-well plate. When they were confluent, the growth medium was removed and commercially available enriched cell culture medium (which includes 1×GHT but no MTX) was added into each well. On the day after adding the commercially available enriched cell culture medium, the plate was incubated at 33° C. for 5-6 days before performing an ELISA assay to quantitate the amount of humanized monoclonal antibody produced by the cells. ELISA was typically performed with serial dilutions of the commercially available enriched cell culture medium. Results from a humanized monoclonal antibody production were shown in FIGS. 9, 10-1, 10-2 and 11.

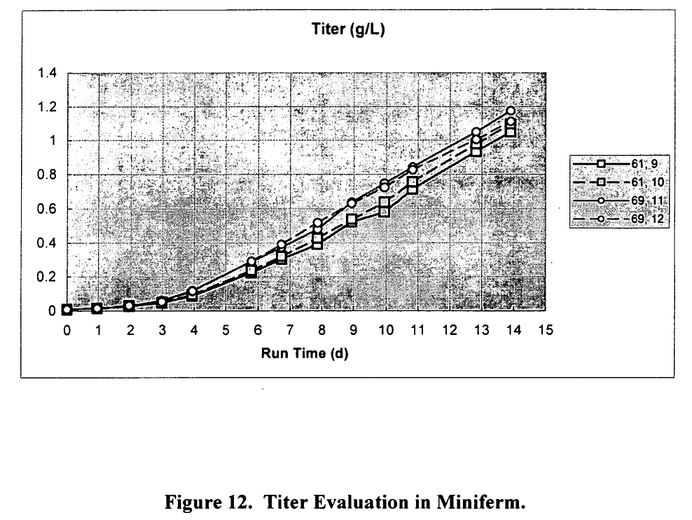

The four clones producing the greatest amount over 100 μg/ml of intact IgG based on direct selection at 25 nM MTX using a CMV-based construct were scaled up from a 96-well plate to a 6-well plate and then to a 10 cm plate. Cells were seeded at 3×10\hat{ }5/ml in 200 ml volume in a 250 ml spinner vessel in serum-free growth medium with 2 μg/ml human insulin and 1× Trace Elements (TE). Cells were initially passaged at either two- or three-day intervals with medium exchange. Then they were passaged at either three- or four-day intervals for about 6 weeks before bioreactor evaluation. At each passage time, cell viability and count number were monitored. To determine the cell growth after serum-free adaptation, a spinner vessel growth experiment was performed. Cells were seeded at 3×10\hat{ }5 cells/ml into 400 ml of growth medium with 2 μg/ml recombinant human insulin and 1×TE in a 500 ml spinner vessel on day 1. On each day, packed cell volume (PCV) was monitored until day 5. PCVs reached between 0.4% to 0.6% by day 4. Two serum-free adapted clones from 25 nM MTX selection with CMV-based construct were evaluated in bioreactors. Two liter bioreactors with commercially available enriched cell culture medium were run for a total of 14 days. The data from the titer evaluation is shown in FIG. 12.

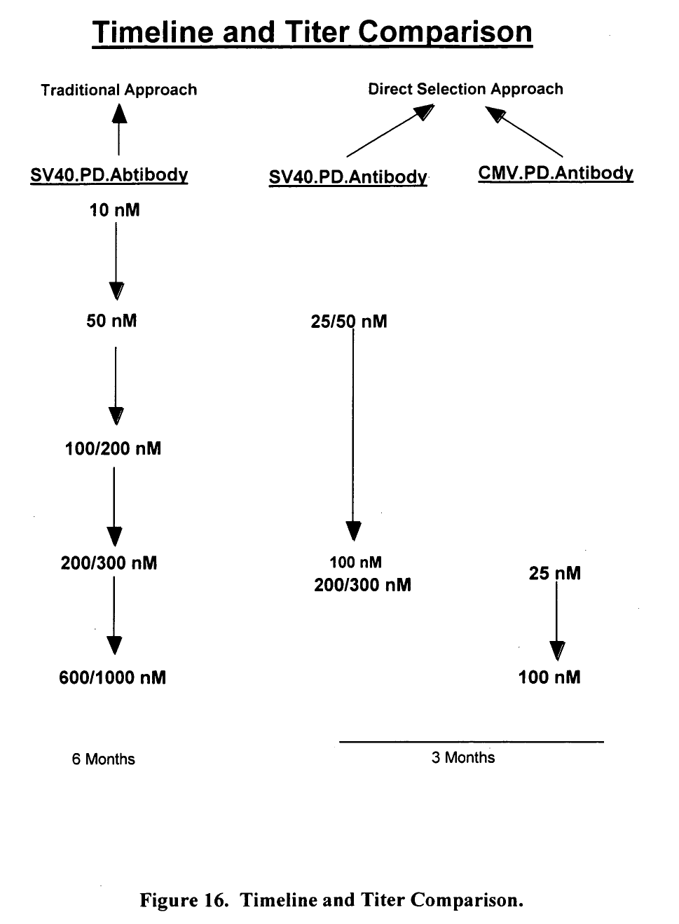

An ELISA assay of clones surviving the direct selection shows that the best clones coming out of the method described in this example produce as much product of interest as highly amplified clones from a traditional method. See FIG. 16. Evaluations of 2 clones from the direct selection shows that those clones produce about 1 g/L of a product of interest in a bioreactor process. Since those clones were generated from one step of a direct selection immediately after transfection, it only takes about 5 to 6 weeks to generate a stable cell line producing 1 g/L of a product of interest in a bioreactor leading to significant timeline reduction, about 3 months, which is critical for efficiency of product development.

The foregoing written specification is considered to be sufficient to enable one skilled in the art to practice the invention. The present invention is not to be limited in scope by the examples presented herein, since the exemplified embodiments are intended as illustrations of certain aspects of the invention and any functionally equivalent embodiments are within the scope of this invention. The examples presented herein are not intended as limiting the scope of the claims to the specific illustrations. Indeed, various modifications of the invention, in addition to those shown and described herein and which fall within the scope of the appended claims, may become apparent to those skilled in the art from the foregoing description.

1-20. (canceled)

21. A method of producing a host cell capable of producing a product of interest, comprising:
(a) introducing a DNA construct into a population of host cells, wherein the DNA construct comprises, in operable linkage, a transcriptional regulatory region; a fusion gene comprising a selectable gene and an amplifiable gene; and a gene encoding the product of interest;
(b) culturing the host cell population in a selective medium, wherein the culturing is the first exposure of the host cell culture to selective conditions; and
(c) cloning a host cell from the selected host cell population, wherein the host cell is capable of producing at least about 250 mg/l of the product of interest.

22. The method of claim 21, wherein the fusion gene is positioned within an intron between the transcriptional regulatory region and the gene encoding the product of interest, the intron defined by a 5′ splice donor site and a 3′ splice acceptor site.

23. The method of claim 22, wherein the intron provides a splicing efficiency of between 80% and 99%.

24. The method of claim 22, wherein the amplifiable gene is the gene encoding DHFR and the selective medium comprises at least about 25 nM methotrexate.

25. The method of claim 22, wherein the amplifiable gene is the gene encoding DHFR and the selective medium comprises at least about 50 nM methotrexate.

26. The method of claim 22, wherein the host cell is a mammalian cell.

27. The method of claim 26, wherein the host cell is a CHO cell.

28. The method of claim 22, wherein the amplifiable gene is selected from the group consisting of the gene encoding dihydrofolate reductase (DHFR) and the gene encoding glutamine synthetase.

29. The method of claim 28, wherein the amplifiable gene is the gene encoding DHFR.

30. The method of claim 22, wherein the selectable gene is a gene encoding puromycin resistance.

31. The method of claim 22, wherein the fusion gene comprises a gene encoding puromycin resistance fused to a gene encoding DHFR.

32. The method of claim 31, wherein the gene encoding puromycin resistance is 5′ to the gene encoding DHFR.

33. The method of claim 22, wherein the product of interest is a protein selected from the group consisting of an antibody, enzyme, hormone, lipoprotein, clotting factor, anti-clotting factor, cytokine, viral antigen, chimeric protein, transport protein, regulatory protein, homing receptor, and addressing; or a fragment of said protein.

34. The method of claim 22, wherein said product of interest is a humanized antibody.

35. The method of claim 22, wherein the DNA construct further comprises, in operable linkage, a second transcriptional regulatory region and a second gene encoding a second product of interest.

36. A host cell produced according to the method of claim 22.

37. The host cell of claim 36, wherein the amplifiable gene is the gene encoding DHFR, the selectable gene is a gene encoding puromycin resistance, and the CHO cell has a DHFR- phenotype.

38. A method of producing a product of interest, comprising culturing a host cell produced according to the method of claim 22, under conditions suitable to cause expression of at least about 250 mg/l of the product of interest.

39. A cell culture composition comprising a host cell produced according to claim 22 and at least about 250 mg/l of the product of interest.

40. A method of producing a host cell capable of producing a product of interest comprising introducing a DNA construct into a population of host cells in suspension culture, wherein the DNA construct comprises in order from 5′ to 3′:
a) a transcriptional regulatory region;
b) a transcriptional initiation site;
c) a fusion gene comprising a selectable gene and an amplifiable gene, wherein the fusion gene is positioned within an intron defined by a 5′ splice donor site and a 3′ splice acceptor site;
d) a gene encoding the product of interest; and
e) a transcriptional termination site;
wherein the transcriptional regulatory region regulates transcription of the amplifiable gene and the gene encoding the product of interest.

41. The method of claim 40, further comprising culturing the host cell population in a selective medium, wherein the culturing is the first exposure of the host cell culture to selective conditions.

42. The method of claim 41, further comprising cloning a host cell from the selected host cell population, wherein the host cell is capable of producing at least about 250 mg/l of the product of interest.

43. The method of claim 40, wherein the host cell is capable of producing at least about 250 mg/l of the product of interest.

44. The method of claim 40, wherein the host cell population is in a spinner vessel.

45. The method of claim 40, wherein the host cell culture has a cell density of at least about 5×10⁵/ml.

46. The method of claim 40, wherein the host cell culture has a cell density of at least about 1.5×10⁵/ml.

47. The method of claim 40, wherein the amplifiable gene is the gene encoding DHFR.

48. The method of claim 40, wherein the fusion gene comprises a gene encoding puromycin resistance fused to a gene encoding DHFR.

49. The method of claim 48, wherein the gene encoding puromycin resistance is 5′ to the gene encoding DHFR.

50. The method of claim 40, wherein the product of interest is selected from the group consisting of an antibody, enzyme, hormone, lipoprotein, clotting factor, anti-clotting factor, cytokine, viral antigen, chimeric protein, transport protein, regulatory protein, homing receptor, and addressin and a fragment of any of said product of interest.

51. The method of claim 40, wherein the transcriptional regulatory region comprises a SV40 promoter.

52. The method of claim 40, wherein the transcriptional regulatory region comprises a CMV promoter.

53. The method of claim 40, wherein steps (b) and (c) are performed simultaneously.

54. The method of claim 40, wherein the DNA construct further comprises, in order from 5′ to 3′:
(f) a second transcriptional regulator region;
(g) a second transcriptional initiation site;
(h) a second product gene encoding a second product of interest; and
(i) a transcriptional termination site;
wherein the second transcriptional regulatory region regulates transcription of the second gene encoding the second product of interest.

55. A method of rapidly selecting a host cell producing a product of interest, comprising:
(a) introducing a DNA construct into a population of host cells, wherein the DNA construct comprises, in operable linkage, a transcriptional regulatory region; a fusion gene comprising a selectable gene and an amplifiable gene; and a gene encoding the product of interest;
(b) culturing the host cell population in a selective medium, wherein the culturing is the first exposure of the host cell culture to selective conditions; and
(c) cloning a host cell from the selected host cell population.

56. The method of claim 55, wherein the rapid selection occurs in five to six weeks.


SEQUENCE LISTING (6 sequences)

SEQ ID NO: 1 - DNA, 5147 bp, Artificial sequence
  plasmid pSV.IPUR circular ds-DNA
ttcgagctcgcccgacattgattattgactagagtcgatcgacagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttatccggccgggaacggtgcattggaacgcggattccccgtgccaagagtgacgtaagtaccgcctatagagcgactagtccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgagttaactgctcccctcctaaagctatgcatttttataagaccatgggacttttgctggctttagatccccttggcttcgttagaacgcagctacaattaatacataaccttatgtatcatacacatacgatttaggtgacactatagataacatccactttgcctttctctccacaggtgtccactcccaggtccaactgcacctcggttctatcgattgaattccccggggatcctctagagtcgacctgcagaagcttcgatggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttacctcgagcggccgcttaattaaggcgcgccatttaaatcctgcaggtaacagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagtattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggtattgttaaagtgtgtcctttgtcgatactggtactaatgcttaatt

SEQ ID NO: 2 - DNA, 5171 bp, Artificial sequence
  plasmid pSV.ID circular ds-DNA
ttcgagctcgcccgacattgattattgactagagtcgatcgacagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttatccggccgggaacggtgcattggaacgcggattccccgtgccaagagtgacgtaagtaccgcctatagagtctataggcccaccccttggctctagagagatataagcctaggattttatccccggtgccatcatggttcgaccattgaactgcatcgtcgccgtgtcccaaaatatggggattggcaagaacggagacctaccctgccctccgctcaggaacgcgttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccaccttagactctttgtgacaaggatcatgcaggaatttgaaagtgacacgtttttcccagaaattgatttggggaaatataaacctctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagacccccttggcttcgttagaacgcggctacaattaatacataaccttatgtatcatacacatagatttaggtgacactatagaataacatccactttgcctttctctccacaggtgtcactccaggtcaactgcacctcggttctatcgattgaattccccggggatcctctagagtcgacctgcagaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttacctcgagcggccgcttaattaaggcgcgccatttaaatcctgcaggtaacagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagtattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaattaa

SEQ ID NO: 3 - DNA, 5712 bp, Artificial sequence
  plasmid pSV.IPD circular ds-DNA
ttcgagctcgcccgacattgattattgactagagtcgatcgacagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttatccggccgggaacggtgcattggaacgcggattccccgtgccaagagtgacgtaagtaccgcctatagagcgactagtccaccatgaccgagtacaagcccacggtgcgcctcgccacccgcgacgacgtcccgcgggccgtacgcaccctcgccgccgcgttcgccgactaccccgccacgcgccacaccgtagacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgccaacatggttcgaccattgaactgcatcgtcgccgtgtcccaaaatatggggattggcaagaacggagacctaccctgccctccgctcaggaacgcgttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccaccttagactctttgtgacaaggatcatgcaggaatttgaaagtgacacgtttttcccagaaattgatttggggaaatataaacctctcccagaatacccaggcgtcctctctgaggtccaggaggaaaaaggcatcaagtataagtttgaagtctacgagaagaaagactaacgttaactgctcccctcctaaagctatgcatttttataagaccatgggacttttgctggctttagatccccttggcttcgttagaacgcagctacaattaatacataaccttatgtatcatacacatacgatttaggtgacactatagataacatccactttgcctttctctccacaggtgtccactcccaggtccaactgcacctcggttctatcgattgaattccccggggatcctctagagtcgacctgcagaagcttcgatggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttacctcgagcggccgcttaattaaggcgcgccatttaaatcctgcaggtaacagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagtattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaattaa

SEQ ID NO: 4 - DNA, 12514 bp, Artificial sequence
  plasmid pSV.IPD.2C4 circular ds-DNA
ttcgagctcgcccgacattgattattgactagagtcgatcgacagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttatccggccgggaacggtgcattggaacgcggattccccgtgccaagagtgacgtaagtaccgcctatagagcgactagtccaccatgaccgagtacaagcccacggtgcgcctcgccacccgcgacgacgtcccgcgggccgtacgcaccctcgccgccgcgttcgccgactaccccgccacgcgccacaccgtagacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgccaacatggttcgaccattgaactgcatcgtcgccgtgtcccaaaatatggggattggcaagaacggagacctaccctgccctccgctcaggaacgcgttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccaccttagactctttgtgacaaggatcatgcaggaatttgaaagtgacacgtttttcccagaaattgatttggggaaatataaacctctcccagaatacccaggcgtcctctctgaggtccaggaggaaaaaggcatcaagtataagtttgaagtctacgagaagaaagactaacgttaactgctcccctcctaaagctatgcatttttataagaccatgggacttttgctggctttagatccccttggcttcgttagaacgcagctacaattaatacataaccttatgtatcatacacatacgatttaggtgacactatagaataacatccactttgcctttctctccacaggtgtccactcccaggtccaactgcacctcggttctatcgattgaattccaccatgggatggtcatgtatcatcctttttctagtagcaactgcaactggagtacattcagaagttcagctggtggagtctggcggtggcctggtgcagccagggggctcactccgtttgtcctgtgcagcttctggcttcaccttcaccgactataccatggactgggtccgtcaggccccgggtaagggcctggaatgggttgcagatgttaatcctaacagtggcggctctatctataaccagcgcttcaagggccgtttcactctgagtgttgacagatctaaaaacacattatacctgcagatgaacagcctgcgtgctgaggacactgccgtctattattgtgctcgtaacctgggaccctctttctactttgactactggggtcaaggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccctagagtcgacctgcagaagcttcgatggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggactaggcttttgcaaaaagctagcttatccggccgggaacggtgcattggaacgcggattccccgtgccaagagtcaggtaagtaccgcctatagagtctataggcccacccccttggcttcgttagaacgcggctacaattaatacataaccttttggatcgatcctactgacactgacatccactttttctttttctccacaggtgtccactcccaggtccaactgcacctcggttcgcgaagctagcttgggctgcatcgattgaattccaccatgggatggtcatgtatcatcctttttctagtagcaactgcaactggagtacattcagatatccagatgacccagtccccgagctccctgtccgcctctgtgggcgatagggtcaccatcacctgcaaggccagtcaggatgtgtctattggtgtcgcctggtatcaacagaaaccaggaaaagctccgaaactactgatttactcggcttcctaccgatacactggagtcccttctcgcttctctggatccggttctgggacggatttcactctgaccatcagcagtctgcagccagaagacttcgcaacttattactgtcaacaatattatatttatccttacacgtttggacagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcttcgatggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataagtttaaaccctctgaaagaggaacttggttaggtaccgactagtagcaaggtcgccacgcacaagatcaatattaacaatcagtcatctctctttagcaataaaaaggtgaaaaattacattttaaaaatgacaccatagacgatgtatgaaaataatctacttggaaataaatctaggcaaagaagtgcaagactgttacccagaaaacttacaaattgtaaatgagaggttagtgaagatttaaatgaatgaagatctaaataaacttataaattgtgagagaaattaatgaatgtctaagttaatgcagaaacggagagacatactatattcatgaactaaaagacttaatattgtgaaggtatactttcttttcacataaatttgtagtcaatatgttcaccccaaaaaagctgtttgttaacttgtcaacctcatttcaaaatgtatatagaaagcccaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagtcattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaggtaccgcggccgcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcccgcgggcaaggtcgccacgcacaagatcaatattaacaatcagtcatctctctttagcaataaaaaggtgaaaaattacattttaaaaatgacaccatagacgatgtatgaaaataatctacttggaaataaatctaggcaaagaagtgcaagactgttacccagaaaacttacaaattgtaaatgagaggttagtgaagatttaaatgaatgaagatctaaataaacttataaattgtgagagaaattaatgaatgtctaagttaatgcagaaacggagagacatactatattcatgaactaaaagacttaatattgtgaaggtatactttcttttcacataaatttgtagtcaatatgttcaccccaaaaaagctgtttgttaacttgtcaacctcatttcaaaatgtatatagaaagcccaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagtcattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattaa

SEQ ID NO: 5 - DNA, 6782 bp, Artificial sequence
  plasmid pCMV.IPD.HP circular ds-DNA
ttcgagctcgcccgacattgattattgactagagtcgatcaccggtagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctgggcccggccgaggccgcctcggcctctgagctattccagaagtagtgaggaggcttttttggaggcctaggcttttgcaaaaagctagcttatccggccgggaacggtgcattggaacgcggattccccgtgccaagagtgacgtaagtaccgcctatagagcgactagtccaccatgaccgagtacaagcccacggtgcgcctcgccacccgcgacgacgtcccgcgggccgtacgcaccctcgccgccgcgttcgccgactaccccgccacgcgccacaccgtagacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccaacatggttcgaccattgaactgcatcgtcgccgtgtcccaaaatatggggattggcaagaacggagacctaccctggcctccgctcaggaacgcgttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacctctcccagaatacccaggcgtcctctctgaggtccaggaggaaaaaggcatcaagtataagtttgaagtctacgagaagaaagactaacgttaactgctcccctcctaaagctatgcatttttataagaccatgagacttttgctggctttagatccccttggcttcgttagaacgcagctacaattaatacataaccttatgtatcatacacatacgatttaggtgacactatagaataacatccactttgcctttctctccacaggtgtccactcccaggtccaactgcacctcggttctatcgattgaattccacccgatggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataacctctgaaagaggaacttggttaggtacctattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctgctagcttatccggccgggaacggtgcattggaacgcggattccccgtgccaagagtcaggtaagtaccgcctatagagtctataggcccacccccttggcttcgttagaacgcggctacaattaatacataaccttttggatcgatcctactgacactgacatccactttttctttttctccacaggtgtccactcccaggtccaactgcacctcggttcgcgaagctcgcttgggctgcatcgattgaattccaccatgggatggtcatgtatcatcctttttctcgatggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataagtttaaaccctctgaaagaggaacttggttaggtaccgactagtcttttgcaaaaagctgttacctcgagcggccgcttaattaaggcgcgccatttaaatcctgcaggtaacagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacaccgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattaa

SEQ ID NO: 6 - DNA, 6124 bp, Artificial sequence
  Plasmid pSV.IPD.H P circular ds-DNA
ttcgagctcgcccgacattgattattgactagagtcgatcgacagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttatccggccgggaacggtgcattggaacgcggattccccgtgccaagagtgacgtaagtaccgcctatagagcgactagtccaccatgaccgagtacaagcccacggtgcgcctcgccacccgcgacgacgtcccgcgggccgtacgcaccctcgccgccgcgttcgccgactaccccgccacgcgccacaccgtagacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgccaacatggttcgaccattgaactgcatcgtcgccgtgtcccaaaatatggggattggcaagaacggagacctaccctgccctccgctcaggaacgcgttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccaccttagactctttgtgacaaggatcatgcaggaatttgaaagtgacacgtttttcccagaaattgatttggggaaatataaacctctcccagaatacccaggcgtcctctctgaggtccaggaggaaaaaggcatcaagtataagtttgaagtctacgagaagaaagactaacgttaactgctcccctcctaaagctatgcatttttataagaccatgggacttttgctggctttagatccccttggcttcgttagaacgcagctacaattaatacataaccttatgtatcatacacatacgatttaggtgacactatagataacatccactttgcctttctctccacaggtgtccactcccaggtccaactgcacctcggttctatcgattgaattccacccgatggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggagcttttgcaaaaagctagcttatccggccgggaacggtgcattggaacgcggattccccgtgccaagagtcaggtaagtaccgcctatagagtctataggcccacccccttggcttcgttagaacgcggctacaattaatacataaccttttggatcgatcctactgacactgacatccactttttctttttctccacaggtgtccactcccaggtccaactgcacctcggttcgcgaagctagcttgggctgcatcgattgaattccacccgatggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataagtttaaaccctctgaaagaggaacttggttaggtaccgactagtcttttgcaaaaagctgttacctcgagcggccgcttaattaaggcgcgccatttaaatcctgcaggtaacagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattaa